(19) United States
(12) Patent Application Publication
(10) Pub. No.: US 2025/0145996 A1
(43) Pub. Date: May 8, 2025

(54) OLIGONUCLEOTIDES TARGETING RNA BINDING PROTEIN SITES

(71) Applicant: Hoffmann-La Roche Inc., Little Falls, NJ (US)

(72) Inventors: Jessica Marine Aurore BASTIEN, Basel (CH); Katarzyna CHYZYNSKA, Charlottenlund (DK); Lars JOENSON, Viby Sjaelland (DK); Meiling LI, Rheinfelden (CH); Bettina NORDBO, Skaevinge (DK); Jonas VIKESAA, Fredensborg (DK)

(21) Appl. No.: 18/948,897

(22) Filed: Nov. 15, 2024

Related U.S. Application Data
(63) Continuation of application No. PCT/EP2023/063442, filed on May 18, 2023.

(30) Foreign Application Priority Data
May 18, 2022 (EP) 22174186.1

(51) Int. Cl. C12N 15/113

(52) U.S. Cl. CPC C12N 15/113; C12N 2310/11; C12N 2310/315; C12N 2310/321; C12N 2310/322; C12N 2310/3231; C12N 2310/3233

(57) ABSTRACT

The present invention relates to antisense oligonucleotides which are complementary to conserved TDP-43 binding sites on pre-mRNA transcripts, which are capable of restoring RNA binding protein function in the processing of multiple independent mRNAs in TDP-43 depleted cells. The contiguous nucleotide sequence of the antisense oligonucleotides comprise 2′-O-methoxyethyl-RNA (2′-MOE) nucleosides and the antisense oligonucleotides are attached to cholesterol moieties.

### SEQUENCE LISTING

The instant application contains a Sequence Listing which has been submitted electronically in XML format and is hereby incorporated by reference in its entirety. Said XML copy, created on Dec. 17, 2024, is named UPDATED P124383US Sequence Listing_ST26.xml and is 139,553 bytes in size.

### FIELD OF INVENTION

The present invention relates to antisense oligonucleotides which are complementary, such as fully complementary, to RNA binding protein target sites on multiple RNAs, such as TDP-43 binding sites on multiple RNA transcripts, and are capable of restoring RNA binding protein functionality to the multiple RNA transcript, such as for use in conditions and medical indications where the RNA binding protein is functionally depleted. The contiguous nucleotide sequence of the antisense oligonucleotides comprise one or more 2′-O-methoxyethyl-RNA (2′-MOE) nucleosides and the antisense oligonucleotides are attached to at least one cholesterol moiety.

### BACKGROUND

TAR DNA binding protein 43 (TDP-43) is a versatile RNA/DNA binding protein involved in RNA-related metabolism. Dysregulation of TDP-43 deposits act as inclusion bodies in the brain and spinal cord of patients with the motor neuron diseases: amyotrophic lateral sclerosis (ALS) and frontotemporal lobar degeneration (FTLD) (Prasad et al., Front. Mol. Neurosci., 2019).

TDP-43 is predominantly localized in the nucleus but also shuttles to the cytoplasm for some of its functions (Ayala et al., 2008). In disease, such as in ALS and FTLD, there is an increase in the cytoplasmic TDP-43 concentration leading to cytoplasmic inclusion formation (Neumann et al., 2006; Winton et al., 2008a). The cytoplasmic mislocalization can be associated with nuclear depletion, resulting in a reduction or loss of TDP-43 function. There are TDP-43 mutations which result in aberrant splicing of TDP-43 target RNAs, resulting in widespread splicing aberration (see for example Arnold et al., PNAS 2013 110 E736-745 and Yang et al., PNAS. U.S.A. 111, E1121-E1129).

Klim et al., reports that STMN2 loss upon reduced TDP-43 function is due to altered STMN2 splicing, and suggests restoring STMN2 as a therapeutic strategy for ALS.

TDP-43 depletion is indicated in a range of diseases, referred to as TDP-43 pathologies, and including for example diseases such as amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinson's disease, autism, Hippocampal sclerosis dementia, Down syndrome, Huntington's disease, polyglutamine diseases, such as spinocerebellar ataxia 3, myopathies and Chronic Traumatic Encephalopathy.

Tollervey et al., Nature Neuroscience 2010, 452-458 reports on the characterization of the RNA targets and position dependent splicing regulation of TDP-43 in healthy brain tissue and brain tissue from FTLD patients. Most TDP-43 binding sites mapped to introns, long non-coding RNAs (IncRNA) and intergenic transcripts, which were enriched for UG-rich motifs. The conserved RNP segments in TDP-43 are involved in binding to TAR DNA sequences and RNA sequences with UG-repeats (Ayala et al., J. Mol. Biol. 2005; 348:575-588). TDP-43 depletion in cells, such as in TDP-pathologies is correlated to the loss of RNA binding of TDP-43 to TDP-43 RNA targets.

TDP-43 binding sites in human RNAs are available on line from the A database of RNA binding proteins and associated moTifs-see https://attract.cnic.es/results/e9129380-8921-406e-84a8. 27ce9b9398b4 #. Certain characterized human RNA TDP-43 binding sites disclosed include the following RNA sequences: GUGAAUGA, GUUGUGC, UGUGUGUGUGUG (SEQ ID NO 85), GAAUGG, UGUGUGUG, GAAUGA, UGUGUG, GUUGUUC, and GUUUUGC.

Melamed et al., reports on premature polyadenylation-mediated loss of STMN2 as a hallmark of TDP-43 neurodegeneration. WO2019/241648 discloses 2′O-methoxyethyl ASOs for increasing STMN2 expression.

The present inventors have identified antisense oligonucleotides which are complementary, such as fully complementary to TDP-43 nucleic acid binding sites and are capable of restoring the processing or regulation of TDP-43 RNA transcripts targets, e.g. the expression and splicing of RNA transcripts, that are dysregulated in cells showing TDP-43 loss of function, and thereby provides a novel approach to restore TDP-43 functionality in TDP-43 depleted cells (i.e. cells with a loss of TDP-43 function), as well as a novel therapeutic approach for the treatment of TDP-43 pathologies.

The inventors have determined that it is possible, through modifications, to reduce the toxicity of antisense oligonucleotides whilst maintaining or improving potency.

### Objective of the Invention

The present invention relates to antisense oligonucleotides which are complementary to conserved TDP-43 binding sites on pre-mRNA transcripts, which are capable of restoring RNA binding protein function in the processing of multiple independent mRNAs in TDP-43 depleted cells. The contiguous nucleotide sequence of the antisense oligonucleotides comprise one or more 2′-O-methoxyethyl-RNA (2′-MOE) nucleosides and the antisense oligonucleotides are attached to at least one cholesterol moiety.

The present invention provides antisense oligonucleotides for restoring RNA binding protein functionality, such as TDP-43 functionality, or TDP-43 like functionality, in cells which have a reduced level of functioning TDP-43.

The present invention provides oligonucleotide which are capable of restoring the nuclear function of TDP-43 in the RNA processing or expression of one or more TDP-43 target RNAs, and thereby restore, at least partially, or enhance, the functional phenotype of the TDP-43 target RNA(s). Such oligonucleotide compounds are referred to herein as RNA binding protein mimics, such as TDP-43 mimics.

The present invention provides antisense oligonucleotides which are complementary to TDP-43 binding sites, and their use in therapy, such as for the treatment of TDP-43 pathologies.

The present invention further provides antisense oligonucleotides which are complementary to TDP-43 binding sites on multiple RNA transcripts, i.e. RNA transcripts which are transcribed from distinct genetic loci. The multiple RNA transcripts may, for example, be independently selected from the group consisting of pre-mRNAs, mRNAs, and IncRNA.

### SUMMARY OF INVENTION

The invention provides an antisense oligonucleotide of 8 to 40 nucleotides in length, which comprises a contiguous nucleotide sequence of at least 8 nucleotides in length which is complementary to a sequence selected from the group consisting of (5′-3′) (UG) n, (GU) n, wherein n is 4-20, UGUGUGUG, UGUGUGUGU, UGUGUGUGUG (SEQ ID NO 95), UGUGUGUGUGU (SEQ ID NO 84), UGUGUGUGUGUG (SEQ ID NO 85), UGUGUGUGUGUGU (SEQ ID NO 86), GUGUGUGU, GUGUGUGUG, GUGUGUGUGU (SEQ ID NO 87), GUGUGUGUGUG (SEQ ID NO 88), GUGUGUGUGUGU (SEQ ID NO 89), GUGUGUGUGUGUG (SEQ ID NO 90), and GUGAAUGA, wherein the contiguous nucleotide sequence comprises one or more 2′-O-methoxyethyl-RNA (2′-MOE) nucleosides and wherein the antisense oligonucleotide is attached to at least one cholesterol moiety.

In some embodiments the antisense oligonucleotide, or contiguous nucleotide sequence thereof, may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32, 33, 34, 35, 36, 37, 38, 39 or 40 2′-O-methoxyethyl-RNA (2′-MOE) nucleosides.

In some embodiments the antisense oligonucleotide, or contiguous nucleotide sequence thereof, may comprise at least 10%, at least 20%, at least 30%, at least 40%, at least 50%, at least 60%, at least 70%, at least 80%, at least 90% or 100% 2′-O-methoxyethyl-RNA (2′-MOE) nucleosides.

In some embodiments all of the nucleosides of the contiguous nucleotide sequence, or contiguous sequence thereof, may be 2′-O-methoxyethyl-RNA (2′-MOE) nucleosides.

In some embodiments the antisense oligonucleotide may be attached to two or more or three or more cholesterol moieties. In some embodiments the antisense oligonucleotide may be attached to one cholesterol moiety. In some embodiments the antisense oligonucleotide may be attached to two cholesterol moieties. In some embodiments the antisense oligonucleotide may be attached to three cholesterol moieties.

In some embodiments the cholesterol moiety or cholesterol moieties may be covalently attached to the antisense oligonucleotide.

In some embodiments the cholesterol moiety may be selected from the group comprising: 5′-cholesterol-TEG-CE phosphoramidite, 5′-cholesterol-CE phosphoramidite or cholesteryl-TEG-CE phosphoramidite. In some embodiments the cholesterol moieties may be a combination selected from the group comprising: 5′-cholesterol-TEG-CE phosphoramidite, 5′-cholesterol-CE phosphoramidite or cholesteryl-TEG-CE phosphoramidite. In some embodiments the cholesterol moiety is 5′-cholesterol-TEG-CE phosphoramidite. In some embodiments all of the cholesterol moieties are 5′-cholesterol-TEG-CE phosphoramidite.

In embodiments where more than one cholesterol moiety is present, each cholesterol moiety is independently selected such that the cholesterol moieties within an antisense oligonucleotide may or may not be the same.

In some embodiments a linker may be positioned between the antisense oligonucleotide and the cholesterol moiety. In some embodiments a linker may be positioned between the antisense oligonucleotide and each of the cholesterol moieties. In some embodiments a linker may be positioned between each of the cholesterol moieties.

In some embodiments the linker may be a cleavable linker.

In some embodiments the linker may be a physiologically labile linker, such as a S1 nuclease susceptible linker.

In some embodiments an alkyl group linker is positioned between the antisense oligonucleotide and the cholesterol moiety or cholesterol moieties.

In some embodiments the physiologically labile linker is a phosphodiester linked cytidine-adenosine dinucleotide with three consecutive phosphodiester linkages.

In some embodiments a C3 alkyl group is a linker positioned between the antisense oligonucleotide and the cholesterol moiety or cholesterol moieties. In some embodiments a C3 alkyl group is positioned between the antisense oligonucleotide and another linker.

In some embodiments a C6 alkyl group is a linker positioned between the antisense oligonucleotide and the cholesterol moiety or cholesterol moieties. In some embodiments a C6 alkyl group is positioned between the antisense oligonucleotide and another linker.

In some embodiments a C12 alkyl group is a linker positioned between the antisense oligonucleotide and the cholesterol moiety or cholesterol moieties. In some embodiments a C12 alkyl group is positioned between the antisense oligonucleotide and another linker.

In some embodiments a TEG group is a linker positioned between the antisense oligonucleotide and the cholesterol moiety or cholesterol moieties. In some embodiments another TEG group is positioned between the antisense oligonucleotide and another linker.

In some embodiments a HEG group is a linker positioned between the antisense oligonucleotide and the cholesterol moiety or cholesterol moieties. In some embodiments a HEG group is positioned between the antisense oligonucleotide and another linker. In some embodiments the antisense oligonucleotide may contain a phosphorothioate (PS) or a phosphodiester (PO) linkage between the cholesterol moiety and the linker.

In another embodiments the antisense oligonucleotide may contain a phosphorothioate (PS) or a phosphodiester (PO) linkage between the linker and the antisense oligonucleotide.

In another embodiment the antisense oligonucleotide may contain a phosphorothioate (PS) or a phosphodiester (PO) linkage between the cholesterol moiety and the linker and a phosphorothioate (PS) or a phosphodiester (PO) linkage between the linker and the antisense oligonucleotide. For example, in some embodiments the antisense oligonucleotide may have the structure: “cholesterol-PO/PS-linker-PO/PS-oligonucleotide”, wherein a PO/PS group is positioned between the linker and the cholesterol moiety and another PO/PS group is positioned between the linker and the antisense oligonucleotide. Here the phosphorothioate (PS) or a phosphodiester (PO) linkages may be independently selected so that both are phosphorothioate (PS) linkages, both are phosphodiester (PO) linkages or one is a phosphorothioate (PS) linkage and the other is a phosphodiester (PO) linkage.

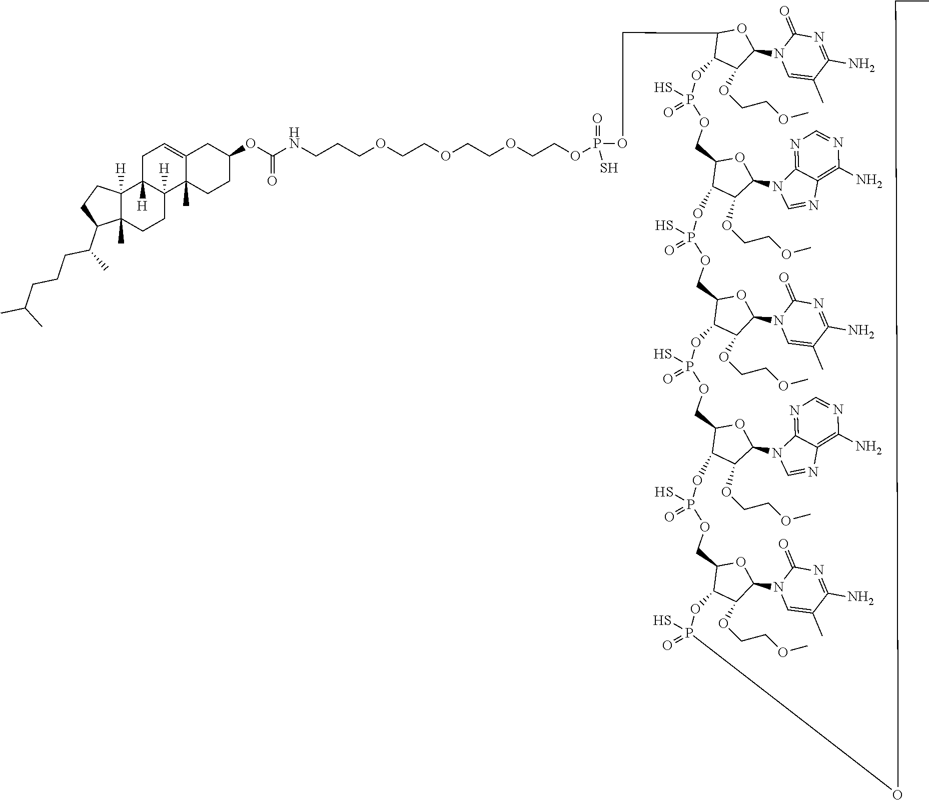

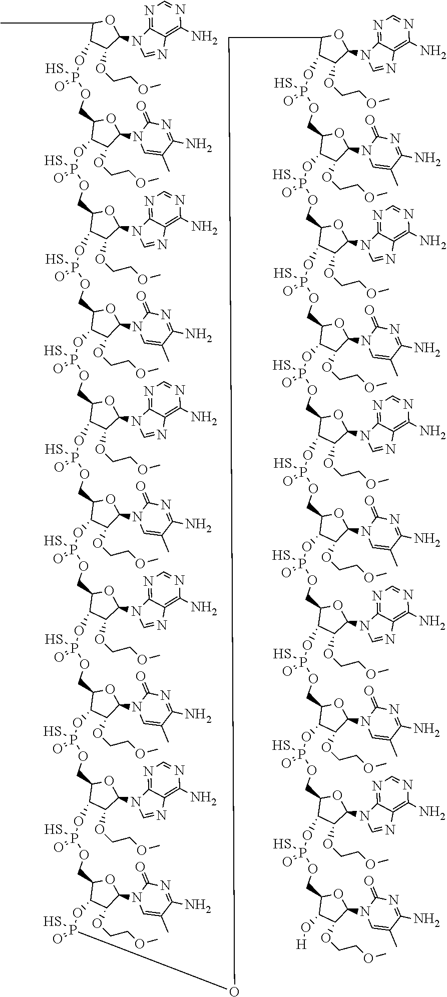

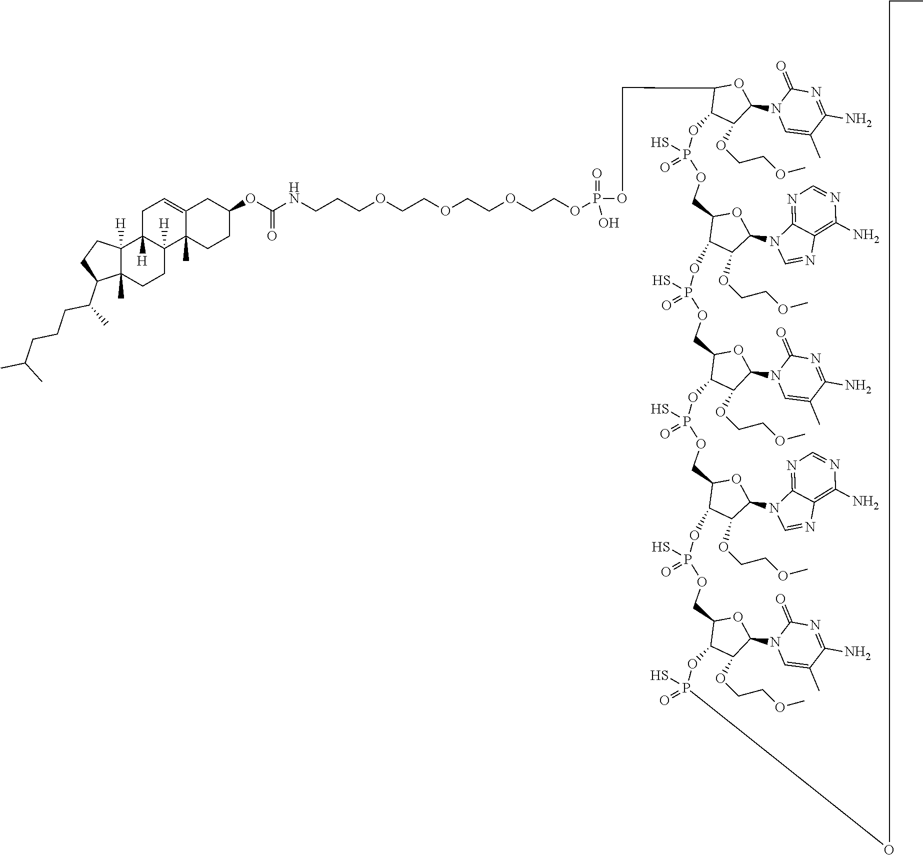

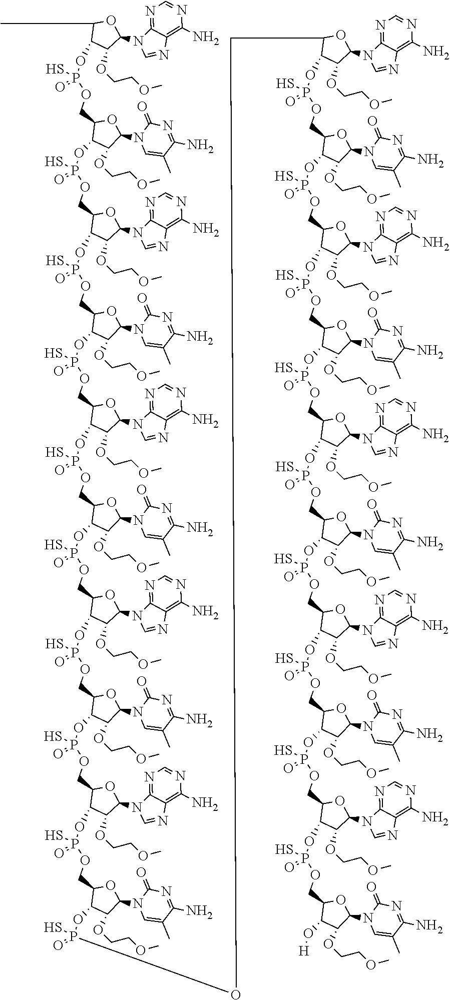

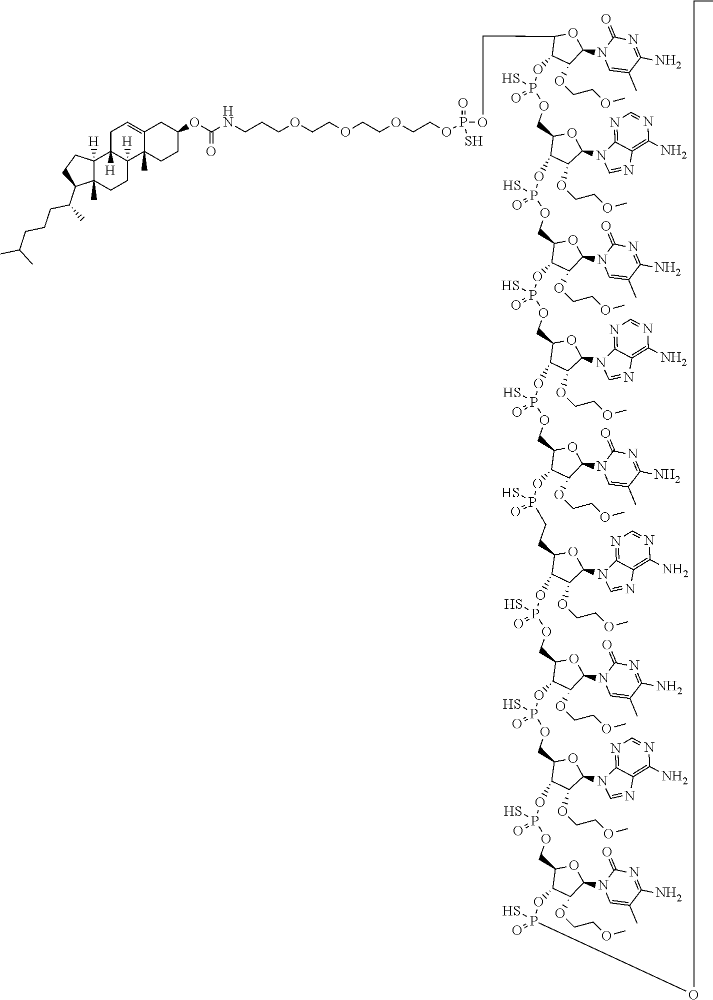

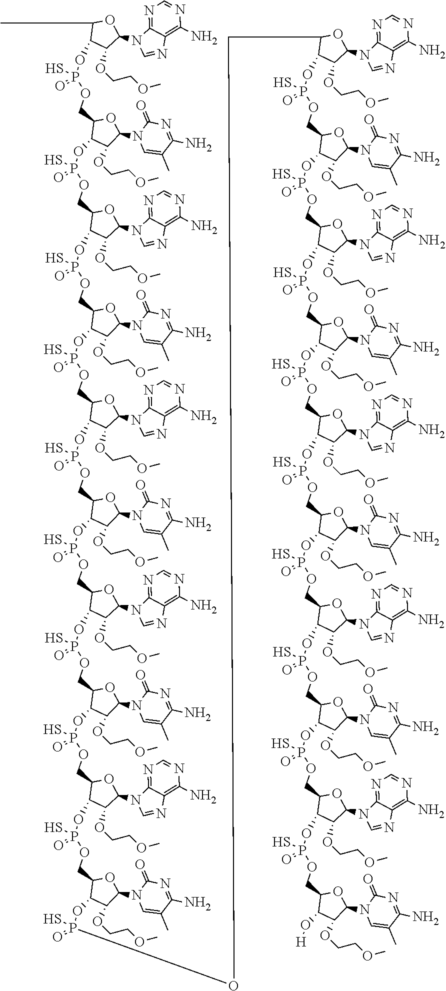

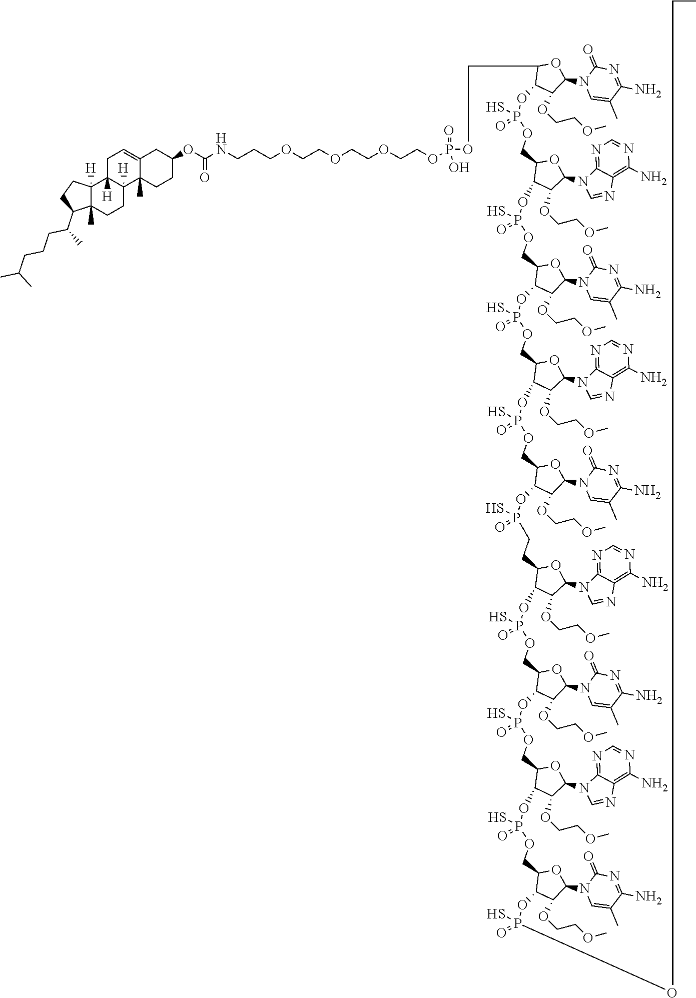

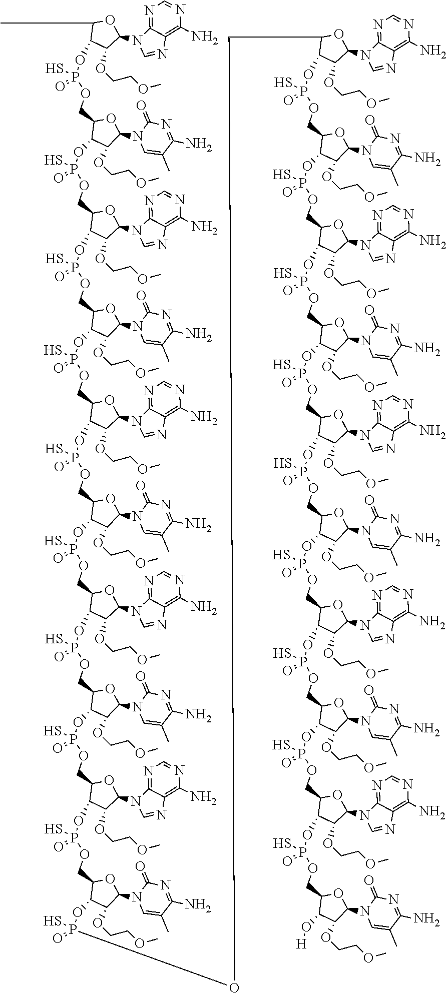

As explained in the background section, functional TDP-43 is primarily a nuclear localized protein, which may exist in the cytoplasm. However, aggregation of TDP-43 in the cytoplasm, what are referred to as cytoplasmic inclusions (also referred to as aberrant TDP-43), is associated with non-functional TDP-43, and this is associated with a loss of nuclear TDP-43 functionality for example in the processing of numerous pre-mRNAs. Cells which express TDP-43 in cytoplasmic inclusions are therefore to be considered TDP-43 depleted.

The antisense oligonucleotide may be an isolated antisense oligonucleotide or a purified oligonucleotide. The antisense oligonucleotide of the invention is a manufactured (man made) antisense oligonucleotide.

The functional phenotype may for example be RNA processing events which are modulated by or dependent upon functional TDP-43 (i.e. non-aberrant TDP-43, typically nuclear TDP-43), and/or whose fidelity is dependent upon functional TDP-43. The enhancement of TDP-43 functionality by use of the antisense oligonucleotides of the invention may therefore be evaluated by assessing the fidelity of RNA processing events which are modulated by or dependent upon functional TDP-43, as for example illustrated herein, with reference to the STMN2, CAMK2B, KALRN, ACTL6B and UNC13A RNA processing.

Advantageously, the antisense oligonucleotide, or contiguous nucleotide sequence thereof, comprises at least 12 or at least 13 contiguous nucleotides, which are complementary, such as fully complementary, to the sequence UGUGUGUGUGUG (SEQ ID NO 85), or GUGUGUGUGUGU (SEQ ID NO 89), or UGUGUGUGUGUGU (SEQ ID NO 86), or GUGUGUGUGUGUG (SEQ ID NO 90).

In some embodiments, the antisense oligonucleotide, or contiguous nucleotide sequence thereof, comprises at least 14 contiguous nucleotides which are complementary, such as fully complementary, to the sequence UGUGUGUGUGUGUG (SEQ ID NO 91), or GUGUGUGUGUGUGU (SEQ ID NO 92).

In some embodiments, the antisense oligonucleotide, or contiguous nucleotide sequence thereof, comprises at least 18 contiguous nucleotides which are complementary, such as fully complementary, to the sequence (UG) n or (GU) n, wherein n is an integer 6-20, such as 7-9.

In some embodiments, the antisense oligonucleotide, or contiguous nucleotide sequence thereof, comprises at least 18 contiguous nucleotides which are complementary, such as fully complementary, to the sequence UGUGUGUGUGUGUGUGUG (SEQ ID NO 93), or GUGUGUGUGUGUGUGUGU (SEQ ID NO 94).

In some embodiments, the antisense oligonucleotide according to the invention, or the contiguous nucleotide sequence thereof, comprises the sequence selected from CACACAC, CACACACA, CACACACAC, ACACACAC, or ACACACACA.

In some embodiments, the antisense oligonucleotide according to the invention, or the contiguous nucleotide sequence thereof, comprises the sequence of SEQ ID NO 1.

In some embodiments, the antisense oligonucleotide according to the invention, or the contiguous nucleotide sequence thereof, comprises the sequence of SEQ ID NO 2.

In some embodiments, the antisense oligonucleotide according to the invention, or the contiguous nucleotide sequence thereof, comprises the sequence of SEQ ID NO 3.

In some embodiments, the antisense oligonucleotide according to the invention, or the contiguous nucleotide sequence thereof, comprises the sequence of SEQ ID NO 4.

In some embodiments, the antisense oligonucleotide according to the invention, or the contiguous nucleotide sequence thereof, comprises the sequence of SEQ ID NO 5.

In some embodiments, the antisense oligonucleotide according to the invention, or the contiguous nucleotide sequence thereof, comprises the sequence of SEQ ID NO 6.

In some embodiments, the antisense oligonucleotide according to the invention, or the contiguous nucleotide sequence thereof, comprises the sequence of SEQ ID NO 7.

In some embodiments, the antisense oligonucleotide according to the invention, or the contiguous nucleotide sequence thereof, comprises the sequence of SEQ ID NO 8.

In some embodiments, the antisense oligonucleotide according to the invention, or the contiguous nucleotide sequence thereof, comprises the sequence of SEQ ID NO 9.

In some embodiments, the antisense oligonucleotide according to the invention, or the contiguous nucleotide sequence thereof, comprises the sequence of SEQ ID NO 10.

In some embodiments, the antisense oligonucleotide according to the invention, or the contiguous nucleotide sequence thereof, comprises the sequence of SEQ ID NO 11.

In some embodiments, the antisense oligonucleotide according to the invention, or the contiguous nucleotide sequence thereof, comprises the sequence of SEQ ID NO 12.

In some embodiments, the antisense oligonucleotide according to the invention, or the contiguous nucleotide sequence thereof, comprises the sequence of SEQ ID NO 13.

In some embodiments, the antisense oligonucleotide according to the invention, or the contiguous nucleotide sequence thereof, comprises the sequence of SEQ ID NO 14.

In some embodiments, the antisense oligonucleotide according to the invention, or the contiguous nucleotide sequence thereof, comprises the sequence SEQ ID NO 15.

In some embodiments, the antisense oligonucleotide according to the invention, or the contiguous nucleotide sequence thereof, comprises the sequence SEQ ID NO 16.

In some embodiments, the antisense oligonucleotide according to the invention, or the contiguous nucleotide sequence thereof, comprises the sequence SEQ ID NO 17.

In some embodiments, the antisense oligonucleotide according to the invention, or the contiguous nucleotide sequence thereof, comprises the sequence SEQ ID NO 18.

In some embodiments, the antisense oligonucleotide according to the invention, or the contiguous nucleotide sequence thereof, comprises the sequence SEQ ID NO 19.

In some embodiments, the antisense oligonucleotide according to the invention, or the contiguous nucleotide sequence thereof, comprises the sequence SEQ ID NO 20.

In some embodiments, the antisense oligonucleotide according to the invention, or the contiguous nucleotide sequence thereof, comprises the sequence SEQ ID NO 21.

In some embodiments, the antisense oligonucleotide according to the invention, or the contiguous nucleotide sequence thereof, comprises the sequence SEQ ID NO 22.

In some embodiments, the antisense oligonucleotide according to the invention, or the contiguous nucleotide sequence thereof, comprises the sequence SEQ ID NO 23.

In some embodiments, the antisense oligonucleotide according to the invention, or the contiguous nucleotide sequence thereof, comprises the sequence SEQ ID NO 24.

In some embodiments, the antisense oligonucleotide according to the invention, or the contiguous nucleotide sequence thereof, comprises the sequence SEQ ID NO 25.

In some embodiments, the antisense oligonucleotide according to the invention, or the contiguous nucleotide sequence thereof, comprises the sequence SEQ ID NO 26.

In some embodiments, the antisense oligonucleotide according to the invention, or the contiguous nucleotide sequence thereof, comprises the sequence SEQ ID NO 27.

In some embodiments, the antisense oligonucleotide according to the invention, or the contiguous nucleotide sequence thereof, comprises the sequence SEQ ID NO 28.

In some embodiments, the antisense oligonucleotide according to the invention, or the contiguous nucleotide sequence thereof, comprises the sequence SEQ ID NO 29.

In some embodiments, the antisense oligonucleotide according to the invention, or the contiguous nucleotide sequence thereof, comprises the sequence SEQ ID NO 30.

In some embodiments, the antisense oligonucleotide according to the invention, or the contiguous nucleotide sequence thereof, comprises the sequence SEQ ID NO 31.

In some embodiments, the antisense oligonucleotide according to the invention, or the contiguous nucleotide sequence thereof, comprises the sequence SEQ ID NO 32.

In some embodiments, the antisense oligonucleotide according to the invention, or the contiguous nucleotide sequence thereof, comprises the sequence SEQ ID NO 33.

In some embodiments, the antisense oligonucleotide according to the invention, or the contiguous nucleotide sequence thereof, comprises the sequence SEQ ID NO 34.

In some embodiments, the antisense oligonucleotide according to the invention, or the contiguous nucleotide sequence thereof, comprises the sequence SEQ ID NO 35.

In some embodiments, the antisense oligonucleotide according to the invention, or the contiguous nucleotide sequence thereof, comprises the sequence SEQ ID NO 36.

In some embodiments, the antisense oligonucleotide according to the invention, or the contiguous nucleotide sequence thereof, comprises the sequence SEQ ID NO 37.

In some embodiments, the antisense oligonucleotide according to the invention, or the contiguous nucleotide sequence thereof, comprises the sequence SEQ ID NO 38.

In some embodiments, the antisense oligonucleotide according to the invention, or the contiguous nucleotide sequence thereof, comprises the sequence SEQ ID NO 39.

In some embodiments, the antisense oligonucleotide according to the invention, or the contiguous nucleotide sequence thereof, comprises the sequence SEQ ID NO 40.

In some embodiments, the antisense oligonucleotide according to the invention, or the contiguous nucleotide sequence thereof, comprises the sequence SEQ ID NO 41.

In some embodiments, the antisense oligonucleotide according to the invention, or the contiguous nucleotide sequence thereof, comprises the sequence SEQ ID NO 42.

In some embodiments, the antisense oligonucleotide according to the invention, or the contiguous nucleotide sequence thereof, comprises the sequence SEQ ID NO 43.

In some embodiments, the antisense oligonucleotide according to the invention, or the contiguous nucleotide sequence thereof, comprises the sequence SEQ ID NO 44.

In some embodiments, the antisense oligonucleotide according to the invention, or the contiguous nucleotide sequence thereof, comprises the sequence SEQ ID NO 45.

In some embodiments, the antisense oligonucleotide according to the invention, or the contiguous nucleotide sequence thereof, comprises the sequence SEQ ID NO 46.

In some embodiments, the antisense oligonucleotide according to the invention, or the contiguous nucleotide sequence thereof, comprises the sequence SEQ ID NO 47.

In some embodiments, the antisense oligonucleotide according to the invention, or the contiguous nucleotide sequence thereof, comprises the sequence SEQ ID NO 48.

In some embodiments, the antisense oligonucleotide according to the invention, or the contiguous nucleotide sequence thereof, comprises the sequence SEQ ID NO 49.

In some embodiments, the antisense oligonucleotide according to the invention, or the contiguous nucleotide sequence thereof, comprises the sequence SEQ ID NO 50.

In some embodiments, the antisense oligonucleotide according to the invention, or the contiguous nucleotide sequence thereof, comprises the sequence SEQ ID NO 51.

In some embodiments, the antisense oligonucleotide according to the invention, or the contiguous nucleotide sequence thereof, comprises the sequence SEQ ID NO 52.

In some embodiments, the antisense oligonucleotide according to the invention, or the contiguous nucleotide sequence thereof, comprises the sequence SEQ ID NO 53.

In some embodiments, the antisense oligonucleotide according to the invention, or the contiguous nucleotide sequence thereof, comprises the sequence SEQ ID NO 54.

In some embodiments, the antisense oligonucleotide according to the invention, or the contiguous nucleotide sequence thereof, comprises the sequence SEQ ID NO 55.

In some embodiments, the antisense oligonucleotide according to the invention, or the contiguous nucleotide sequence thereof, comprises the sequence SEQ ID NO 56.

In some embodiments, the antisense oligonucleotide according to the invention, or the contiguous nucleotide sequence thereof, comprises the sequence SEQ ID NO 57.

In some embodiments, the antisense oligonucleotide according to the invention, or the contiguous nucleotide sequence thereof, comprises the sequence SEQ ID NO 58.

In some embodiments, the antisense oligonucleotide according to the invention, or the contiguous nucleotide sequence thereof, comprises the sequence SEQ ID NO 59.

In some embodiments, the antisense oligonucleotide according to the invention, or the contiguous nucleotide sequence thereof, comprises the sequence SEQ ID NO 60.

In some embodiments, the antisense oligonucleotide according to the invention, or the contiguous nucleotide sequence thereof, comprises the sequence SEQ ID NO 61.

In some embodiments, the antisense oligonucleotide according to the invention, or the contiguous nucleotide sequence thereof, comprises the sequence SEQ ID NO 62.

In some embodiments, the antisense oligonucleotide according to the invention, or the contiguous nucleotide sequence thereof, comprises the sequence SEQ ID NO 63.

In some embodiments, the antisense oligonucleotide according to the invention, or the contiguous nucleotide sequence thereof, comprises the sequence SEQ ID NO 64.

In some embodiments, the antisense oligonucleotide according to the invention, or the contiguous nucleotide sequence thereof, comprises the sequence SEQ ID NO 65.

In some embodiments, the antisense oligonucleotide according to the invention, or the contiguous nucleotide sequence thereof, comprises the sequence SEQ ID NO 66.

In some embodiments, the antisense oligonucleotide according to the invention, or the contiguous nucleotide sequence thereof, comprises the sequence SEQ ID NO 67.

In some embodiments, the antisense oligonucleotide according to the invention, or the contiguous nucleotide sequence thereof, comprises the sequence SEQ ID NO 68.

In some embodiments, the antisense oligonucleotide according to the invention, or the contiguous nucleotide sequence thereof, comprises the sequence SEQ ID NO 69.

In some embodiments, the antisense oligonucleotide according to the invention, or the contiguous nucleotide sequence thereof, comprises the sequence SEQ ID NO 70.

In some embodiments, the antisense oligonucleotide according to the invention, or the contiguous nucleotide sequence thereof, comprises the sequence SEQ ID NO 71.

In some embodiments, the antisense oligonucleotide according to the invention, or the contiguous nucleotide sequence thereof, comprises the sequence SEQ ID NO 72.

In some embodiments, the antisense oligonucleotide according to the invention, or the contiguous nucleotide sequence thereof, comprises the sequence SEQ ID NO 73.

In some embodiments, the antisense oligonucleotide according to the invention, or the contiguous nucleotide sequence thereof, comprises the sequence SEQ ID NO 74.

In some embodiments, the antisense oligonucleotide according to the invention, or the contiguous nucleotide sequence thereof, comprises the sequence SEQ ID NO 75.

In some embodiments, the antisense oligonucleotide according to the invention, or the contiguous nucleotide sequence thereof, comprises the sequence SEQ ID NO 76.

In some embodiments, the antisense oligonucleotide according to the invention, or the contiguous nucleotide sequence thereof, comprises the sequence SEQ ID NO 77.

In some embodiments, the antisense oligonucleotide according to the invention, or the contiguous nucleotide sequence thereof, comprises the sequence SEQ ID NO 78.

In some embodiments, the antisense oligonucleotide according to the invention, or the contiguous nucleotide sequence thereof, comprises the sequence SEQ ID NO 79.

In some embodiments, the antisense oligonucleotide according to the invention, or the contiguous nucleotide sequence thereof, comprises the sequence SEQ ID NO 80.

In some embodiments, the antisense oligonucleotide according to the invention, or the contiguous nucleotide sequence thereof, comprises the sequence SEQ ID NO 81.

In some embodiments, the antisense oligonucleotide according to the invention, or the contiguous nucleotide sequence thereof, comprises the sequence SEQ ID NO 82.

In some embodiments, the antisense oligonucleotide according to the invention, or the contiguous nucleotide sequence thereof, comprises the sequence SEQ ID NO 83.

In some embodiments, the antisense oligonucleotide according to the invention, or the contiguous nucleotide sequence thereof, comprises a sequence or compound as displayed in Table 1.

In some embodiments, the antisense oligonucleotide according to the invention, or the contiguous nucleotide sequence thereof, comprises SEQ ID NO 118-126.

In some embodiments, the antisense oligonucleotide according to the invention, or the contiguous nucleotide sequence thereof, comprises SEQ ID NO 118.

In some embodiments, the antisense oligonucleotide according to the invention, or the contiguous nucleotide sequence thereof, comprises SEQ ID NO 119.

In some embodiments, the antisense oligonucleotide according to the invention, or the contiguous nucleotide sequence thereof, comprises SEQ ID NO 120.

In some embodiments, the antisense oligonucleotide according to the invention, or the contiguous nucleotide sequence thereof, comprises SEQ ID NO 121.

In some embodiments, the antisense oligonucleotide according to the invention, or the contiguous nucleotide sequence thereof, comprises SEQ ID NO 122.

In some embodiments, the antisense oligonucleotide according to the invention, or the contiguous nucleotide sequence thereof, comprises SEQ ID NO 123.

In some embodiments, the antisense oligonucleotide according to the invention, or the contiguous nucleotide sequence thereof, comprises SEQ ID NO 124.

In some embodiments, the antisense oligonucleotide according to the invention, or the contiguous nucleotide sequence thereof, comprises SEQ ID NO 125.

In some embodiments, the antisense oligonucleotide according to the invention, or the contiguous nucleotide sequence thereof, comprises SEQ ID NO 126.

In some embodiments, the antisense oligonucleotide according to the invention, or the contiguous nucleotide sequence thereof, comprises the sequence (CA) n or (AC) n, wherein n is an integer 1-20, such as 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-15, 1-10, 1-5, 5-15, 5-10 or 10-15.

In some embodiments, the antisense oligonucleotide according to the invention, or the contiguous nucleotide sequence thereof, comprises the sequence (CA) n or (AC) n, wherein n is an integer such as 1, 2, 3, 4, 5, 6, 7, 8, 9, 10, 11, 12, 13, 14, 15, 16, 17, 18, 19 or 20.

In some embodiments, the antisense oligonucleotide according to the invention, or the contiguous nucleotide sequence thereof, comprises a sequence selected from the group consisting of SEQ ID No 1-83 or SEQ ID No 118-126.

In some embodiments, the antisense oligonucleotide according to the invention, or the contiguous nucleotide sequence thereof, consists of a sequence selected from the group consisting of SEQ ID No 1-83 or SEQ ID No 118-126.

The contiguous nucleotide sequence may comprise a fragment of 8 or more contiguous nucleotides of any one of SEQ ID No 1-83 or SEQ ID No 118-126.

The contiguous nucleotide sequence may consist of a fragment of 8 or more contiguous nucleotides of any one of SEQ ID No 1-83 or SEQ ID No 118-126.

In some embodiments the antisense oligonucleotide or contiguous nucleotide sequence thereof according to any aspect of the invention has a length of at least 8 contiguous nucleotides.

In some embodiments the antisense oligonucleotide or contiguous nucleotide sequence thereof according to any aspect of the invention has a length of at least 9 contiguous nucleotides.

In some embodiments the antisense oligonucleotide or contiguous nucleotide sequence thereof according to any aspect of the invention has a length of at least 10 contiguous nucleotides.

In some embodiments the antisense oligonucleotide or contiguous nucleotide sequence thereof according to any aspect of the invention has a length of at least 11 contiguous nucleotides.

In some embodiments the antisense oligonucleotide or contiguous nucleotide sequence thereof according to any aspect of the invention has a length of at least 12 contiguous nucleotides.

In some embodiments the antisense oligonucleotide or contiguous nucleotide sequence thereof according to any aspect of the invention has a length of at least 13 contiguous nucleotides.

In some embodiments the antisense oligonucleotide or contiguous nucleotide sequence thereof according to any aspect of the invention has a length of at least 14 contiguous nucleotides.

In some embodiments the antisense oligonucleotide or contiguous nucleotide sequence thereof according to any aspect of the invention has a length of at least 15 contiguous nucleotides.

In some embodiments the antisense oligonucleotide or contiguous nucleotide sequence thereof according to any aspect of the invention has a length of at least 16 contiguous nucleotides.

In some embodiments the antisense oligonucleotide or contiguous nucleotide sequence thereof according to any aspect of the invention has a length of at least 17 contiguous nucleotides.

In some embodiments the antisense oligonucleotide or contiguous nucleotide sequence thereof according to any aspect of the invention has a length of at least 18 contiguous nucleotides.

In some embodiments the antisense oligonucleotide or contiguous nucleotide sequence thereof according to any aspect of the invention has a length of at least 19 contiguous nucleotides.

In some embodiments the antisense oligonucleotide or contiguous nucleotide sequence thereof according to any aspect of the invention has a length of at least 20 contiguous nucleotides.

In some embodiments the antisense oligonucleotide or contiguous nucleotide sequence thereof according to any aspect of the invention has a length of at least 21 contiguous nucleotides.

In some embodiments the antisense oligonucleotide or contiguous nucleotide sequence thereof according to any aspect of the invention has a length of at least 22 contiguous nucleotides.

In some embodiments the antisense oligonucleotide or contiguous nucleotide sequence thereof according to any aspect of the invention has a length of at least 23 contiguous nucleotides.

In some embodiments the antisense oligonucleotide or contiguous nucleotide sequence thereof according to any aspect of the invention has a length of at least 24 contiguous nucleotides.

In some embodiments the antisense oligonucleotide or contiguous nucleotide sequence thereof according to any aspect of the invention has a length of at least 25 contiguous nucleotides.

In some embodiments the antisense oligonucleotide or contiguous nucleotide sequence thereof according to any aspect of the invention has a length of at least 26 contiguous nucleotides.

In some embodiments the antisense oligonucleotide or contiguous nucleotide sequence thereof according to any aspect of the invention has a length of at least 27 contiguous nucleotides.

In some embodiments the antisense oligonucleotide or contiguous nucleotide sequence thereof according to any aspect of the invention has a length of at least 28 contiguous nucleotides.

In some embodiments the antisense oligonucleotide or contiguous nucleotide sequence thereof according to any aspect of the invention has a length of at least 29 contiguous nucleotides.

In some embodiments the antisense oligonucleotide or contiguous nucleotide sequence thereof according to any aspect of the invention has a length of at least 30 contiguous nucleotides.

In some embodiments the antisense oligonucleotide or contiguous nucleotide sequence thereof according to any aspect of the invention has a length of at least 31 contiguous nucleotides.

In some embodiments the antisense oligonucleotide or contiguous nucleotide sequence thereof according to any aspect of the invention has a length of at least 32 contiguous nucleotides.

In some embodiments the antisense oligonucleotide or contiguous nucleotide sequence thereof according to any aspect of the invention has a length of at least 33 contiguous nucleotides.

In some embodiments the antisense oligonucleotide or contiguous nucleotide sequence thereof according to any aspect of the invention has a length of at least 34 contiguous nucleotides.

In some embodiments the antisense oligonucleotide or contiguous nucleotide sequence thereof according to any aspect of the invention has a length of at least 35 contiguous nucleotides.

In some embodiments the antisense oligonucleotide or contiguous nucleotide sequence thereof according to any aspect of the invention has a length of at least 36 contiguous nucleotides.

In some embodiments the antisense oligonucleotide or contiguous nucleotide sequence thereof according to any aspect of the invention has a length of at least 37 contiguous nucleotides.

In some embodiments the antisense oligonucleotide or contiguous nucleotide sequence thereof according to any aspect of the invention has a length of at least 38 contiguous nucleotides.

In some embodiments the antisense oligonucleotide or contiguous nucleotide sequence thereof according to any aspect of the invention has a length of at least 39 contiguous nucleotides.

In some embodiments the antisense oligonucleotide or contiguous nucleotide sequence thereof according to any aspect of the invention has a length of at least 40 contiguous nucleotides.

In some embodiments the antisense oligonucleotide may consists of the contiguous nucleotide sequence.

In some embodiments the antisense oligonucleotide may be the contiguous nucleotide sequence.

In some embodiments, the contiguous nucleotide sequence may be at least 75% complementary to the target sequence.

In some embodiments, the contiguous nucleotide sequence may be at least 80%, at least 81%, at least 82%, at least 83%, at least 84%, at least 85%, at least 86%, at least 87%, at least 88%, at least 89%, at least 90% at least 91%, at least 92%, at least 93%, at least 94%, at least 95%, at least 96%, at least 97%, at least 98%, or at least 99% complementary to the target sequence.

In some embodiments, the contiguous nucleotide sequence may comprise 1, 2, 3, 4, 5, 6, 7, 8 or more mismatches to the target sequence.

In some embodiments, the antisense oligonucleotide according to the invention has a Gibbs free energy of the antisense oligonucleotide to a complementary target RNA, of lower than about −10AG, such as lower than about −15 AG, such as lower than about −17 AG.

The antisense oligonucleotide may be capable of restoring the functional phenotype of one or more TDP-43 target RNA(s) in a cell which is TDP-43 depleted, or is expressing aberrant TDP-43 protein.

Advantageously the antisense oligonucleotide of the invention, which comprises a contiguous nucleotide sequence comprising one or more 2′-O-methoxyethyl-RNA (2′-MOE) nucleosides, may further comprise one or more additionally or alternatively modified nucleosides. In other words, the antisense oligonucleotide of the invention comprises one or more 2′-MOE nucleosides and may further comprise one or more additionally or alternatively modified nucleosides.

Advantageously the antisense oligonucleotide of the invention may comprise LNA nucleosides. LNA nucleotides within the contiguous nucleotide sequence are further advantageous. In some embodiments, the antisense oligonucleotide of the invention may comprise LNA nucleosides and non-LNA nucleosides, such as DNA nucleosides. In some embodiments, the antisense oligonucleotide, or contiguous nucleotide sequence thereof, may comprise LNA and DNA nucleosides. In some embodiments, all of the nucleosides of the antisense oligonucleotide, or contiguous nucleotide sequence thereof, are independently selected from LNA and DNA nucleosides. Advantageously, the length of contiguous DNA nucleosides present within the antisense oligonucleotide, or contiguous nucleotide sequence thereof, is limited so as to prevent RNaseH recruitment which results in target RNA degradation. Suitably the antisense oligonucleotide or contiguous nucleotide sequence thereof does not comprise more than four contiguous DNA nucleosides, more advantageously does not comprise more than 3 contiguous DNA nucleosides.

When used, advantageously, the antisense oligonucleotide according to the invention is capable of modulating the splicing of two or more TDP-43 target pre-mRNAs (target RNAs). By way of example, the two or more TDP-43 target RNAs may be independently selected from the group consisting of STMN2 pre-mRNA, CAMK2B pre-mRNA, KALRN pre-mRNA, ACTL6B pre-mRNA and UNC13A pre-mRNA.

In some embodiments the antisense oligonucleotide according to the invention is capable of modulating the splicing of one or more TDP-43 target pre-mRNAs (target RNAs).

In some embodiments the antisense oligonucleotide according to the invention is capable of modulating the splicing of two or more TDP-43 target pre-mRNAs (target RNAs).

In some embodiments the antisense oligonucleotide according to the invention is capable of modulating the splicing of three or more TDP-43 target pre-mRNAs (target RNAs).

In some embodiments the antisense oligonucleotide according to the invention is capable of modulating the splicing of four or more TDP-43 target pre-mRNAs (target RNAs).

In some embodiments the antisense oligonucleotide according to the invention is capable of modulating the splicing of one, two, three, four, five, six, seven, eight, nine, ten or more TDP-43 target pre-mRNAs (target RNAs).

In some embodiments, the antisense oligonucleotide is capable of enhancing the fidelity of pre-mRNA splicing of at least one, such as two or more pre-mRNAs selected from the group consisting of STMN2 pre-mRNA CAMK2B pre-mRNA, KALRN pre-mRNA, ACTL6B pre-mRNA and UNC13A pre-mRNA, when administered to a TDP-43 depleted cell. In some embodiments, the two or more selected pre-mRNAs are selected from the groups consisting of STMN2 and CAMK2B; STMN2 and KALRN; STMN2 and ACTL6B; and STMN2 and UNC13A.

In some embodiments, the antisense oligonucleotide is capable of enhancing the fidelity of pre-mRNA splicing of two or more pre-mRNAs selected from the group consisting of STMN2 pre-mRNA, CAMK2B pre-mRNA, KALRN pre-mRNA, ACTL6B pre-mRNA and UNC13A pre-mRNA, when administered to a TDP-43 depleted cell. In some embodiments, the two or more selected pre-mRNAs are selected from the groups consisting of STMN2 and CAMK2B; STMN2 and KALRN; STMN2 and ACTL6B; STMN2 and UNC13A.

In some embodiments, the antisense oligonucleotide is capable of enhancing the fidelity of pre-mRNA splicing of three or more pre-mRNAs selected from the group consisting of STMN2 pre-mRNA, CAMK2B pre-mRNA, KALRN pre-mRNA, ACTL6B pre-mRNA and UNC13A pre-mRNA when administered to a TDP-43 depleted cell.

In some embodiments, the antisense oligonucleotide is capable of enhancing the fidelity of pre-mRNA splicing of STMN2 pre-mRNA, CAMK2B pre-mRNA, KALRN pre-mRNA, ACTL6B pre-mRNA and UNC13A pre-mRNA when administered to a TDP-43 depleted cell.

In some embodiments, the antisense oligonucleotide is capable of decreasing the proportion of STMN2 mature mRNAs which comprise a cryptic exon (ce1) between exon 1 and exon 2, as compared to the wildtype STMN2 mature mRNA with a contiguous exon1/exon2 junction, when administered to a TDP-43 depleted cell which is expressing STMN2 pre-mRNA.

In some embodiments, the antisense oligonucleotide is capable of decreasing the level of aberrant exon inclusion in CAMK2B mRNA transcript, when administered to a TDP-43 depleted cell which is expressing CAMK2B pre-mRNA.

In some embodiments, the antisense oligonucleotide is capable of decreasing the level of aberrant exon inclusion in KALRN mRNA transcript, when administered to a TDP-43 depleted cell which is expressing KALRN pre-mRNA.

In some embodiments, the antisense oligonucleotide is capable of decreasing the level of aberrant exon inclusion in ACTL6B mRNA transcript, when administered to a TDP-43 depleted cell which is expressing ACTL6B pre-mRNA.

In some embodiments, the antisense oligonucleotide is capable of decreasing the level of aberrant exon inclusion in UNC13A mRNA transcript, when administered to a TDP-43 depleted cell which is expressing UNC13A pre-mRNA.

In some embodiments, the antisense oligonucleotide is capable of correcting the aberrant splicing of two or more of STMN2, CAMK2B, KALRN, ACTL6B and UNC13A pre-mRNA in a TDP-43 depleted cell.

In some embodiments, the antisense oligonucleotide does not comprise a region of more than 3, or more than 4, contiguous DNA nucleosides.

In some embodiments, the antisense oligonucleotide, is not capable of mediating RNAseH cleavage.

In some embodiments, the antisense oligonucleotide is a morpholino antisense oligonucleotide.

In some embodiments, the antisense oligonucleotide, or contiguous nucleotide sequence thereof which comprises one or more 2′-MOE nucleosides, may further comprise one or more affinity enhancing nucleosides, such as 2′sugar modified nucleosides which enhance the binding affinity between the antisense oligonucleotide and a complementary RNA molecule, for example and advantageously, to provide a lower Gibbs free energy, such as a Gibbs free energy lower −10, such as lower than −15.

In some embodiments, the antisense oligonucleotide or contiguous nucleotide sequence thereof which comprises one or more 2′-MOE nucleosides, may further comprise one or more modified nucleosides, such as a 2′ sugar modified nucleoside independently selected from the group consisting of: 2′-O-alkyl-RNA; 2′-O-methyl RNA (2′-OMe); 2′-alkoxy-RNA; 2′-amino-DNA; 2′-fluro-RNA; 2′-fluoro-DNA; arabino nucleic acid (ANA); 2′-fluoro-ANA; locked nucleic acid (LNA), or a combination thereof.

In some embodiments, the 2′ sugar modified nucleoside may be an affinity enhancing 2′ sugar modified nucleoside.

In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32, 33, 34, 35, 36, 37, 38, 39 or 40 nucleosides in the antisense oligonucleotide, or contiguous nucleotide sequence thereof, are 2′-O-methoxyethyl-RNA (2′-MOE) nucleosides, optionally linked by one or more phosphorothioate internucleoside linkages.

In some embodiments, at least 10%, at least 20%, at least 30%, at least 40%, at least 50%, at least 60%, at least 70%, at least 80%, at least 90% or 100% of the nucleosides in the antisense oligonucleotide, or contiguous nucleotide sequence thereof, are 2′-O-methoxyethyl-RNA (2′-MOE) nucleosides, optionally linked by one or more phosphorothioate internucleoside linkages.

In some embodiments, all the nucleosides in the antisense oligonucleotide, or contiguous nucleotide sequence thereof, are 2′-O-methoxyethyl-RNA (2′-MOE) nucleosides, optionally linked by one or more phosphorothioate internucleoside linkages.

In some embodiments the antisense oligonucleotide, or contiguous nucleotide sequence thereof which comprises one or more 2′-MOE nucleosides, may further comprise 2′-O-methy nucleosides.

In some embodiments, one or more of the further modified nucleosides within the antisense oligonucleotide, or contiguous nucleotide sequence thereof, is a locked nucleic acid nucleoside (LNA), such as an LNA nucleoside selected from the group consisting of constrained ethyl nucleoside (cEt), or β-D-oxy-LNA.

In some embodiments, the contiguous nucleotide sequence of the antisense oligonucleotide comprises of nucleosides LNA nucleosides and DNA nucleosides, optionally linked by phosphorothioate internucleoside linkages.

In some embodiments, the antisense oligonucleotide or contiguous nucleotide sequence thereof is a mixmer or a totalmer.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 8 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 9 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 10 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 11 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 12 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 13 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 14 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 15 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 16 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 17 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 18 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 19 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 20 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 21 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 22 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 23 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 24 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 25 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 26 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 27 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 28 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 29 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 30 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 31 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 32 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 33 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 34 contiguous nucleotides thereof.

In some embodiments, the antisense oligonucleotide or contiguous nucleotide sequence thereof comprises a sequence of nucleobases selected from the group consisting of SEQ ID Nos 1-83 or SEQ ID Nos 118-126—or at least 35 contiguous nucleotides thereof.

In some embodiments, the cytosine bases present in the antisense oligonucleotide or contiguous nucleotide sequence thereof are independently selected from the group consisting of cytosine and 5-methyl cytosine.

In some embodiments, the cytosine bases present in the antisense oligonucleotide or contiguous nucleotide sequence thereof are 5-methyl cytosine.

In some embodiments, the LNA cytosine bases present in the antisense oligonucleotide or contiguous nucleotide sequence thereof are LNA 5-methyl cytosine.

In some embodiments, the LNA cytosine bases present in the antisense oligonucleotide or contiguous nucleotide sequence thereof are LNA 5-methyl cytosine, and DNA cytosine bases are cytosine.

Advantageously, one or more of the internucleoside linkages positioned between the nucleosides on the contiguous nucleotide sequence are modified. In some embodiments, at least about 75%, at least about 80%, at least about 85%, at least about 90%, at least about 95%, or about 100% of the internucleoside linkages positioned between the nucleosides on the contiguous nucleotide sequence are modified.

In some embodiments, one or more, or all of the modified internucleoside linkages are phosphorothioate linkage. In some embodiments, one or more, or all of the linkages within the contiguous nucleotide sequence, are phosphorothioate linkages.

In some embodiments, all of the internucleoside linkages present in the contiguous nucleotide sequence are phosphorothioate internucleoside linkages.

In some embodiments, all of the internucleoside linkages present in the antisense oligonucleotide are phosphorothioate internucleoside linkages.

In some embodiments, the antisense oligonucleotide of the invention, which is attached to at least one cholesterol moiety, may further comprise one or more conjugate groups. In other words, the antisense oligonucleotide of the invention is attached to at least one cholesterol moiety and may further comprise one or more additional conjugate groups which is not a cholesterol moiety.

In some embodiments, the antisense oligonucleotide of the invention may be covalently attached to at least one conjugate moiety.

In some embodiments, the antisense oligonucleotide of the invention may be in the form of a pharmaceutically acceptable salt. In some embodiments the salt may be a sodium salt or a potassium salt or an ammonium salt.

The invention provides for a pharmaceutical composition comprising the antisense oligonucleotide of the invention and a pharmaceutically acceptable diluent, solvent, carrier, salt and/or adjuvant.

The invention provides for a pharmaceutical composition comprising the antisense oligonucleotide of the invention and a pharmaceutically acceptable diluent or solvent, and a cation. The cation may for example be a sodium cation or a potassium cation. The diluent/solvent maybe water.

The invention provides for a method, such as an in vivo or in vitro method, for enhancing TDP-43 functionality in a cell which is expressing aberrant or depleted levels of TDP-43, said method comprising administering an antisense oligonucleotide of the invention or composition of the invention, in an effective amount to said cell.

The invention provides for a method for treating or preventing a TDP-43 pathology in a subject comprising administering a therapeutically or prophylactically effective amount of an antisense oligonucleotide of the invention or composition of the invention, to a subject suffering from or susceptible to the TDP-43 pathology.

The invention provides for an antisense oligonucleotide of the invention or composition of the invention, for use as a medicament.

The invention provides for an antisense oligonucleotide of the invention or composition of the invention, for use in the treatment of a TDP-43 pathology.

The invention provides for the use of the antisense oligonucleotide of the invention or composition of the invention, for the preparation of a medicament for treatment or prevention of a TDP-43 pathology.

The invention provides for the method of the invention, the antisense oligonucleotide or the pharmaceutical composition for use according to the invention or the use according to the invention, wherein the TDP-43 pathology is a neurological disorder selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

In some embodiments, the TDP-43 pathology is a neurological disorder selected from the group consisting of amyotrophic lateral sclerosis (ALS), frontotemporal lobar degeneration (FTLD).

The invention provides for a pharmaceutical solution comprising the antisense oligonucleotide of the invention and a pharmaceutically acceptable solvent, such as phosphate buffered saline.

In some embodiments, the antisense oligonucleotide of the invention may be in a solid powdered form, such as in the form of a lyophilized powder.

Typically, the antisense oligonucleotide of the invention comprises a contiguous nucleotide sequence of at least 8 or at least 10 nucleotides in length, such as 10-32, 15-32, 20-32, 21-32, 22-32, 23-32, 24-32, 25-32, 26-32, 27-32 or 10-20 nucleotides in length, wherein the contiguous nucleotide sequence is at least 75% complementary, such as at least 90% complementary to or fully complementary to a TDP-43 RNA binding sequence. In some embodiments, all of the nucleosides of the antisense oligonucleotide form the contiguous nucleotide sequence.

In some embodiments, the antisense oligonucleotide of the invention is capable of modulating the splicing of the at least two human pre-mRNAs. For example, the splicing of human STMN2, CAMK2B, KALRN, ACTL6B and UNC13A pre-mRNAs are dependent upon TDP-43 binding, as illustrated in the examples.

In a further aspect the invention provides methods for treating or preventing neurodegenerative disease such as amyotrophic lateral sclerosis (ALS), comprising administering a therapeutically or prophylactically effective amount of the oligonucleotide of the invention to a subject suffering from or susceptible to the disease.

In a further aspect the oligonucleotide or composition of the invention is used for the treatment or prevention of a neurodegenerative disease as neurodegenerative disorders characterized by TDP-43 pathology or mislocalization of TDP-43 from the nucleus, such as amyotrophic lateral sclerosis (ALS).

### SEQUENCE LISTING

The sequence listing submitted with this application is hereby incorporated by reference.

### BRIEF DESCRIPTION OF FIGURES

FIG. 1-Drawing illustrating the linking of cholesterol to the 5′ of ASO.

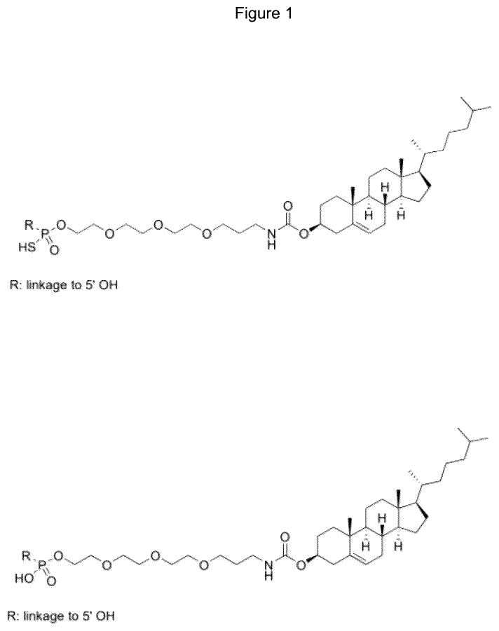

### DETAILED DESCRIPTION OF THE INVENTION

### RNA Binding Protein Mimics and TDP-43 Mimics

TDP-43 is the TAR RNA/DNA binding protein which in humans is encoded on the human chromosome 1:11,012,653-11,022,858 forward strand (Gene ENSG00000120948, Chr 1:11, 012,344-11,025,739, example of a typical TDP-43 transcript=ENST00000439080.6), and is widely involved in RNA splicing, stability and metabolism. In healthy cells the TDP-43 protein is located in the nucleus, however in several neurodegenerative diseases, dysfunction TDP-43 aggregates form in the cytoplasm (often associated with hyper-phosphorylated and ubiquitinated TDP-43).

TDP-43 is an example of a RNA binding protein which binds to GU repeats in numerous independent RNA transcripts. The interaction of RNA binding proteins, such as TDP-43 with the population of numerous RNA transcripts has a profound effect on the biology of the RNA transcripts, such as the splicing on pre-mRNA, RNA stability, RNA accumulation, and therefore provides a mechanism for effecting the expression of populations of independent RNAs in a cell. This is of particular relevance in the case of TDP-43 depletion, where the loss of RNA binding of functional TDP-43 is closely associated with neurodegeneration.

The present invention provides antisense oligonucleotides which are complementary to GU rich regions on multiple RNA transcripts, such as conserved TDP-43 binding sites on a population of pre-mRNA transcripts. As is illustrated in the examples, the administration of the antisense oligonucleotides of the invention can restore the functional processing of multiple independent RNA transcripts, which are otherwise aberrantly processed in the depletion or absence of the RNA binding protein, for example TDP-43. The antisense oligonucleotides of the invention, otherwise referred to as compounds of the invention, may therefore be referred to as RNA binding protein mimics, or TDP-43 mimics, in that they restore the functionality of the RNA binding protein, such as TDP-43, in regulating the RNA biology of multiple RNA transcripts.

By way of example, the RNA binding protein functionality, such as TDP-43 functionality, which is restored or enhanced by the use of the compounds of the invention (e.g. in TDP-43 depleted cells), is the expression, processing, e.g. splicing events of pre-mRNA transcripts, resulting in a restoration of functional gene expression which is otherwise dysregulated in cells with reduced level of functional TDP-43 (referred to herein as TDP-43 depleted cells). This may result in an enhanced gene expression or an enhanced quality of gene expression.

Advantageously, the compounds of the invention are capable of mimicking the functional TDP-43, and restoring the nuclear function of TDP-43 in the expression of one or more TDP-43 target RNAs, and thereby restore the functional phenotype of the TDP-43 target RNA(s).

It will be understood that other RNA binding proteins may bind to the TDP-43 binding sites, and as such the TDP-43 mimics referred to herein are oligonucleotides, which are complementary to the TDP-43 binding sites of one or more RNA targets, such as multiple nucleic acid targets (i.e. RNA targets which are described from distinct genetic loci), and which are capable of restoring the expression of the normal (wildtype)

As reported in Arnold et al., PNAS 2013, some TDP-43 pathologies are associated with certain TDP-43 mutations, and these may not necessarily be associated with TDP-43 cytoplasmic depletion. In the context of the present invention the normal function of TDP-43 may be genetically disrupted, and this is therefore also considered a potential source of depletion or normal TDP-43, a phenotype which can be addressed using the TDP-43 mimics of the present invention.

### Examples of TDP-43 RNA Targets

The depletion of TDP-43 in neuronal cells results in a profound alteration in the RNA processing of a large population of RNA transcripts in the cell.

The examples illustrate five of these TDP-43 target RNAs: STMN2, KALRN, ACTL6B, UNC13A and CAMK2B, but the invention is not limited in this regard.

Arnold et al., PNAS 2013 110 E736-745 identifies widespread aberrations of pre-mRNA splicing of TDP-43 binding RNAs in TDP-43 depleted cells (TDP-43 ASO depleted mice), and illustrates the identification of indicative TDP-43 regulated splicing events using micro-array analysis. RNAs identified by Arnold et al., whose splicing is regulated by TDP-43 include Eif4h, Taf1b, Kcnip2, (TDP-43 mutation dependent), Sort1, Kcnd3, Ahi1, Atxn2, Ctnnd (dose dependent).

STMN2 (Klim et al., Nat Neurosci. 2019 February;22 (2): 167-179)-TDP-43 depletion in neuronal cells (e.g. in ALS) results in a mis-splicing of the STMN2 transcript. STMN2 encodes a microtubule regulator, whose expression declines after TDP-43 knockdown and TDP-43 mislocalization as well as in patient-specific motor neurons and postmortem patient spinal cord. Post-translational stabilization of STMN2 rescued neurite outgrowth and axon regeneration deficits induced by TDP-43 depletion. TDP-43 depletion results in the incorporation of a cryptic intron between exon1 and 2 of STMN2. WO2019/241648 discloses fully MOE modified phosphorothioate ASOs which are used to suppress the mis-splicing of STMN2.

The above mentioned transcripts and the associated TDP-43 depletion splicing events may be used to assay for restoration of TDP-43 functionality using the compounds of the invention.

### TDP-43 Pathologies

A TDP-43 pathology is a disease which is associated with reduced or aberrant expression of TDP-43, often associated by an increase in cytoplasmic TDP-43, particularly hyper-phosphorylated and ubiquitinated TDP-43.

TDP-43 depletion is indicated in a range of diseases, referred to as TDP-43 pathologies, and include for example such as amyotrophic lateral sclerosis (ALS), frontotemporal lobar degeneration (FTLD), Progressive supranuclear palsy (PSP), Primary lateral sclerosis, Progressive muscular atrophy Alzheimer's disease, Parkinson's disease, Autism, Hippocampal sclerosis dementia, Down syndrome, Huntington's disease, polyglutamine diseases, such as spinocerebellar ataxia 3, myopathies and Chronic Traumatic Encephalopathy.

Cells which are Depleted in TDP-43

Cells which are depleted in TDP-43 refer to cells where the functional level of TDP-43 is reduced. It will be understood that in TDP-43 pathologies, aberrant TDP-43 expression results in accumulation of dysfunctional cytoplasmic TDP-43, and a reduction in the functional nuclear

TDP-43 level—as such cells which are depleted in TDP-43 may be characterized by a reduction in the functional level of TDP-43, and may therefore be associated with an increase in the level of dysfunctional TDP-43. For in vitro assessment, TDP-43 depletion may be engineered for example by genetic engineering approaches (e.g. CRISPR/CAS9), or as illustrated in the examples, by use of an antisense oligonucleotide inhibitor of TDP-43 (illustrated by a gapmer oligonucleotide targeting the human TDP-43 transcript).

In some embodiments the cell which is depleted in TDP-43 is a neuronal cell.

### Sequences Complementary to TDP-43 Binding Sites

TDP-43 binding sites are characterized by poly GU motifs (see the A database of RNA binding https://attract.cnic.es/re sie91 80-8921-406e-84a8-27ce9b9398b4 #) for example, and suitably for antisense oligonucleotide intervention may comprise a motif of (GU) n or (UG) n, where n is at least 3 or preferably at least 4. In some embodiments n is 4, 5, 6, 7, 8, 9 or 10.

In some embodiments the TDP-43 binding site may comprise a sequence selected from the group consisting of (UG) n, (GU) n, wherein n is 4-20, UGUGUGUG, UGUGUGUGU, UGUGUGUGUG (SEQ ID NO 95), UGUGUGUGUGU (SEQ ID NO 84), UGUGUGUGUGUG (SEQ ID NO 85), UGUGUGUGUGUGU (SEQ ID NO 86), GUGUGUGU, GUGUGUGUG, GUGUGUGUGU (SEQ ID NO 87), GUGUGUGUGUG (SEQ ID NO 88), GUGUGUGUGUGU (SEQ ID NO 89), GUGUGUGUGUGUG (SEQ ID NO 90), and GUGAAUGA.

In some embodiments, the TDP-43 binding site may comprise a sequence selected from the group consisting of GUGAAUGA, GUUGUGC, UGUGUGUGUGUG (SEQ ID NO 85), GAAUGG, UGUGUGUG, GAAUGA, UGUGUG, GUUGUUC, and GUUUUGC. In some embodiments, the TDP-43 binding site may comprise the sequence UGUGUGUGUGUGUG (SEQ ID NO 91).

In some embodiments the antisense oligonucleotide of the present invention may comprise a sequence which is complementary to, such as fully complementary to one or more sequences selected from the group consisting of (UG) n, (GU) n, wherein n is 4-20, UGUGUGUG, UGUGUGUGU, UGUGUGUGUG (SEQ ID NO 95), UGUGUGUGUGU (SEQ ID NO 84), UGUGUGUGUGUG (SEQ ID NO 85), UGUGUGUGUGUGU (SEQ ID NO 86), GUGUGUGU, GUGUGUGUG, GUGUGUGUGU (SEQ ID NO 87), GUGUGUGUGUG (SEQ ID NO 88), GUGUGUGUGUGU (SEQ ID NO 89), GUGUGUGUGUGUG (SEQ ID NO 90), and GUGAAUGA.

The antisense oligonucleotide of the present invention may comprise a sequence which is complementary to, such as fully complementary to, the TDP-43 binding site sequence, such as one or more sequences, selected from the group consisting of (GU) n, (UG) n, GUGAAUGA, GUUGUGC, GAAUGG, UGUGUGUG, GAAUGA, UGUGUG, UGUGUGUGUGUG (SEQ ID NO 85), GUUGUUC, and GUUUUGC.

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers.

Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The antisense oligonucleotides of the invention are man-made, and are chemically synthesized, and are typically purified or isolated.

The antisense oligonucleotides of the invention, which comprise contiguous nucleotide sequences comprising one or more 2′-MOE nucleosides, may further comprise one or more further or additionally modified nucleosides such as 2′ sugar modified nucleosides. The antisense oligonucleotides of the invention may comprise one or more modified internucleoside linkages, such as one or more phosphorothioate internucleoside linkages.

### Antisense Oligonucleotides

The term “antisense oligonucleotide” as used herein is defined as an oligonucleotide capable of modulating expression of a target gene by hybridizing to a target nucleic acid, in particular to a contiguous sequence on a target nucleic acid. Antisense oligonucleotides are not essentially double stranded and are therefore not siRNAs or shRNAs. The antisense oligonucleotides of the present invention may be single stranded. It is understood that single stranded oligonucleotides of the present invention can form hairpins or intermolecular duplex structures (duplex between two molecules of the same oligonucleotide), as long as the degree of intra or inter self-complementarity is less than approximately 50% across of the full length of the oligonucleotide.

In some embodiments, the single stranded antisense oligonucleotides of the invention may not contain RNA nucleosides.

Advantageously, the antisense oligonucleotides of the invention, which comprise contiguous nucleotide sequences comprising one or more 2′-MOE nucleosides, may further comprise one or more further or additionally modified nucleosides or nucleotides, such as 2′ sugar modified nucleosides. Furthermore, in some antisense oligonucleotides of the invention, it may be advantageous that the nucleosides which are not modified are DNA nucleosides.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleosides of the antisense oligonucleotide constitute the contiguous nucleotide sequence. The contiguous nucleotide sequence is the sequence of nucleotides in the oligonucleotide of the invention which are complementary to, and in some instances fully complementary to, the target nucleic acid or target sequence.

In some embodiments the antisense oligonucleotide comprises the contiguous nucleotide sequence, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo) base moiety. Advantageously, the antisense oligonucleotides of the invention, which comprise contiguous nucleotide sequences comprising one or more 2′-MOE nucleosides, may further comprise one or more further or additionally modified nucleosides which comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing. Exemplary modified nucleosides which may be used in the compounds of the invention include LNA, 2′-O-MOE and morpholino nucleoside analogues.

### Modified Internucleoside Linkage

The term “modified internucleoside linkage” is defined as generally understood by the skilled person as linkages other than phosphodiester (PO) linkages that covalently couple two nucleosides together. The antisense oligonucleotides of the invention may therefore comprise one or more modified internucleoside linkages such as one or more phosphorothioate internucleoside linkage.

In some embodiments at least 50% of the internucleoside linkages in the antisense oligonucleotide, or contiguous nucleotide sequence thereof, are phosphorothioate, such as at least 60%, such as at least 70%, such as at least 75%, such as at least 80% or such as at least 90% or more of the internucleoside linkages in the antisense oligonucleotide, or contiguous nucleotide sequence thereof, are phosphorothioate. In some embodiments all of the internucleoside linkages of the antisense oligonucleotide, or contiguous nucleotide sequence thereof, are phosphorothioate.

Advantageously, all the internucleoside linkages of the contiguous nucleotide sequence of the antisense oligonucleotide are phosphorothioate, or all the internucleoside linkages of the antisense oligonucleotide are phosphorothioate linkages.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro −6-aminopurine, 5′ nitroindole.

The nucleobase moieties may be indicated by the letter code for each corresponding nucleobase, e.g. A, T, G, C or U, wherein each letter may optionally include modified nucleobases of equivalent function. For example, in the exemplified antisense oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an antisense oligonucleotide comprising one or more sugar-modified nucleosides and/or modified internucleoside linkages. The term “chimeric oligonucleotide” is a term that has been used in the literature to describe oligonucleotides comprising sugar modified nucleosides and DNA nucleosides. In some embodiments, it may be advantageous for the antisense oligonucleotide of the invention to be a chimeric oligonucleotide.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which, across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

Within the present invention the level of complementarity between the contiguous nucleotide sequence of the antisense oligonucleotide and the TDP-43 binding site, or target sequence, may be at least about 75%.

Within the present invention the level of complementarity between the contiguous nucleotide sequence of the antisense oligonucleotide and the target TDP-43 binding site, or target sequence, may be at least about 80%.

Within the present invention the level of complementarity between the contiguous nucleotide sequence of the antisense oligonucleotide and the TDP-43 binding site, or target sequence, may be at least about 85%.

Within the present invention the level of complementarity between the contiguous nucleotide sequence of the antisense oligonucleotide and the TDP-43 binding site, or target sequence, may be at least about 90%.

Within the present invention the level of complementarity between the contiguous nucleotide sequence of the antisense oligonucleotide and the TDP-43 binding site, or target sequence, may be at least about 95%.

In some embodiments the contiguous nucleotide sequence may be fully complementary to the TDP-43 binding site, or target sequence. The term “fully complementary”, refers to 100% complementarity.

The compounds of the invention are complementary to TDP-43 binding sites in TDP-43 target RNAs.

Complete complementarity may not be required and in some embodiments, the antisense oligonucleotide may comprise one, two, three, four, five, six, seven, eight or more mismatches to a TDP-43 target RNA TDP-43 RNA binding site to which it effectively binds. In this regard, antisense oligonucleotides maybe designed which are sufficiently complementary to multiple but not identical TDP-43 binding sites in different TDP-43 Target RNAs. In some embodiments an universal base such as inosine may be used in complementary positions in the antisense oligonucleotide where these is not perfect identity of TDP-43 binding site sequence in the multiple TDP-43 RNA targets.

In some embodiments the contiguous nucleotide sequence may include one or more mismatches to a TDP-43 binding site, or target sequence.

In some embodiments the contiguous nucleotide sequence may include two or more mismatches to a TDP-43 binding site, or target sequence.

In some embodiments the contiguous nucleotide sequence may include three or more mismatches to a TDP-43 binding site, or target sequence.

In some embodiments the contiguous nucleotide sequence may include four or more mismatches to a TDP-43 binding site, or target sequence.

In some embodiments the contiguous nucleotide sequence may include five or more mismatches to a TDP-43 binding site, or target sequence.

In some embodiments the contiguous nucleotide sequence may include six or more mismatches to a TDP-43 binding site, or target sequence.

In some embodiments the contiguous nucleotide sequence may include seven or more mismatches to a TDP-43 binding site, or target sequence.

In some embodiments the contiguous nucleotide sequence may include eight or more mismatches to a TDP-43 binding site, or target sequence.

In some embodiments, antisense oligonucleotides of the present invention which contain one more, such as two or more, three or more, four or more, five or more, six or more, seven or more, or eight or more mismatches, may hybridize to a target nucleic acid with estimated ΔG° values below −10 kcal for oligonucleotides that are 10-32 nucleotides in length.

In some embodiments, antisense oligonucleotides of the present invention which contain one more, such as two or more, three or more, four or more, five or more, six or more, seven or more, or eight or more mismatches, may hybridize to a target nucleic acid with estimated ΔG° values below −12 kcal, −15 kcal, −17 kcal, −20 kcal, −30 kcal, −40 kcal, −50 kcal or −60 kcal for oligonucleotides that are 10-32 nucleotides in length.

Calculation of ΔG° values is discussed below.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=-RTIn (K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem.

Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95:1460-1465 using appropriately derived thermodynamic parameters described by Sugimoto et al., 1995, Biochemistry 34:11211-11216 and McTigue et al., 2004, Biochemistry 43:5388-5405. In some embodiments, antisense oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The antisense oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Exemplary TDP-43 RNA Targets

In some embodiments, a TDP-43 target RNA is the mammalian protein known as stathmin 2, or SCG10, SCGN10, for example the human STMN2 as disclosed as Gene: ENSG00000104435 (ensemble.org), encoded on human Chromosome 8:79,610,814-79,666, 175 forward strand (GRCh38: CM000670.2).

In some embodiments, a TDP-43 target RNA is CAMK2B.

In some embodiments, a TDP-43 target RNA is KALRN.

In some embodiments, a TDP-43 target RNA is UNC13A.

In some embodiments, a TDP-43 target RNA is ACTL6B.

### Target Cell

The term “target cell” as used herein refers to a cell which is expressing the targeted TDP-43 RNA targets whose expression is to be corrected by the administration of the compound of the invention. Suitably the target cell is further TDP-43 depleted. For experimental use, TDP-43 depletion may be engineered into the cell, e.g. via genetic engineering (e.g. CRISPR/CAS9) or via the use of ASO inhibitors of TDP-43.

In some embodiments the target cell may be in vivo or in vitro. In some embodiments the target cell is a mammalian cell such as a rodent cell, such as a mouse cell or a rat cell, or a primate cell such as a monkey cell or a human cell.

In some embodiments the target cell is a neuronal cell.

For in vitro evaluation, the target cell may be a glutamatergic neuron (also referred to herein as a glutaneuron cell), such as a human glutamatergic neuron, such as a human glutamatergic neuron which is TDP-43 depleted. Human glutamatergic neuron are available from Cellular Dynamics (iCell GlutaNeurons). For in vitro evaluation the target cell, such as the glutaneuron are in vitro. TDP-43 depletion of the target cell, for example for in vitro evaluation, may be achieved for example using antisense oligonucleotides or siRNA reagents, or may be engineered into the cells e.g. via CRISPR/Cas9 editing, or shRNA vector expression. As further illustrated in the examples, the target cell, for example for in vitro use, may be a human pluripotent stem cell-derived neuron, for example these are obtainable as iCell GlutaNeurons Kit, 01279 Cat. R1034 (Fujifilm Cellular Dynamics).

### Splice Modulation

Splice modulation can be used to correct cryptic splicing, modulate alternative splicing, restore the open reading frame, and induce protein knockdown.

Splice modulation may be assayed by RNA sequencing (RNAseq), which allows for a quantitative assessment of the different splice products of a pre-mRNA, or by digital droplet PCR using PCR assays designed to be specific for one or the other splice form. In some embodiments of the invention, the antisense oligonucleotide modulates the splicing of the STMN2 pre-mRNA, e.g. they reduce the level of mature STMN2 mRNA which comprises a RNA sequence positioned between the exon 1 and exon 2 (as illustrated in the examples), for example in the target cell or TDP-43 depleted cells. In some embodiments of the invention, the antisense oligonucleotide modulates the splicing of the STMN2 pre-mRNA, e.g. they enhance the level of mature correctly spliced STMN2 mRNA which does not comprise a RNA sequence positioned between the exon 1 and exon 2, referred to as WT STMN2 transcript, for example in the target cell.

### High Affinity Modified Nucleosides

A high affinity modified nucleoside is a modified nucleotide which, when incorporated into the antisense oligonucleotide enhances the affinity of the antisense oligonucleotide for its complementary target, for example as measured by the melting temperature (Tm). A high affinity modified nucleoside of the present invention preferably result in an increase in melting temperature between +0.5 to +12° C., more preferably between +1.5 to +10° C. and most preferably between+3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3 (2), 293-213).

### Sugar Modifications

The antisense oligonucleotide of the invention, which comprises a contiguous nucleotide sequence which comprises one or more 2′-MOE nucleosides, may further comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3 (2), 293-213, and Deleavey and Damha,

Chemistry and Biology 2012, 19, 937. Below are illustrations of some 2′ substituted modified nucleosides.

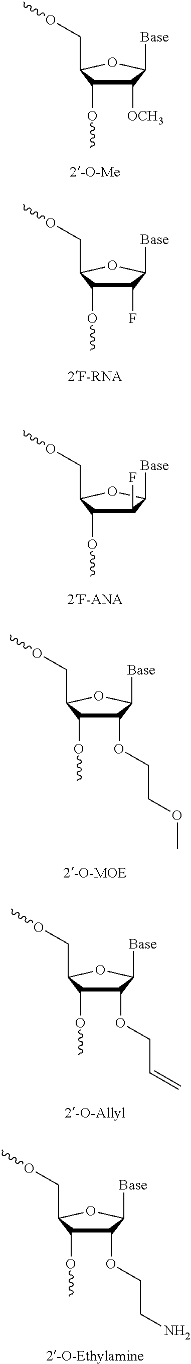

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75 (5) pp. 1569-81, and Mitsuoka et al., Nucleic Acids Research 2009, 37 (4), 1225-1238, and Wan and Seth, J. Medical Chemistry 2016, 59, 9645-9667.

Further non limiting, exemplary LNA nucleosides are disclosed in Scheme 1.

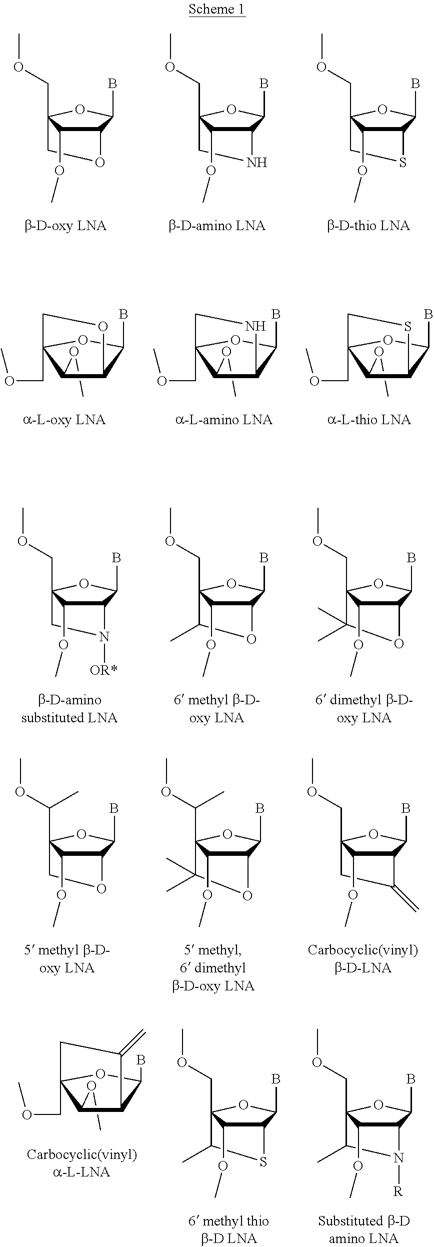

Particular LNA nucleosides are beta-D-oxy-LNA, 6′-methyl-beta-D-oxy LNA such as(S) −6′-methyl-beta-D-oxy-LNA (ScET) and ENA.

A particularly advantageous LNA is beta-D-oxy-LNA.

### Morpholino Oligonucleotides

In some embodiments, the antisense oligonucleotide of the invention comprises or consists of morpholino nucleosides (i.e. is a Morpholino oligomer and as a phosphorodiamidate Morpholino oligomer (PMO)). Splice modulating morpholino oligonucleotides have been approved for clinical use-see for example eteplirsen, a 30nt morpholino oligonucleotide targeting a frame shift mutation in DMD, used to treat Duchenne muscular dystrophy. Morpholino oligonucleotides have nucleobases attached to six membered morpholine rings rather ribose, such as methylenemorpholine rings linked through phosphorodiamidate groups, for example as illustrated by the following illustration of 4 consecutive morpholino nucleotides:

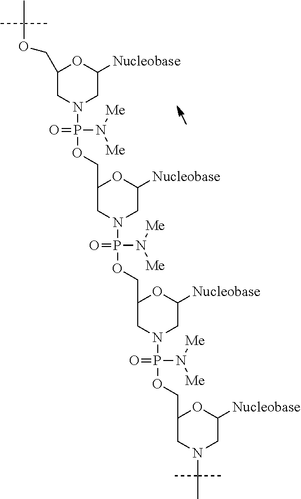

In some embodiments, morpholino oligonucleotides of the invention may be, for example 20-40 morpholino nucleotides in length, such as morpholino 25-35 nucleotides in length.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant RNase H1 is available from Lubio Science GmbH, Lucerne, Switzerland. DNA oligonucleotides are known to effectively recruit RNaseH, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA. For effective modulation of splicing, degradation of the pre-mRNA is not desirable, and as such it is preferable to avoid the RNaseH degradation of the target. Therefore, the antisense oligonucleotide of the invention is preferably not gapmer oligonucleotide. RNaseH recruitment may be avoided by limiting the number of contiguous DNA nucleotides in the antisense oligonucleotide-therefore for effective splice modulation mimxers and totalmers designs may therefore be used.

### Mixmers and Totalmers

For splice modulation it is often advantageous to use antisense oligonucleotides which do not recruit RNAaseH. As RNaseH activity requires a contiguous sequence of DNA nucleotides, RNaseH activity of antisense oligonucleotide may be achieved by designing antisense oligonucleotides which do not comprise a region of more than 3 or more than 4 contiguous DNA nucleosides. This may be achieved by using antisense oligonucleotides or contiguous nucleoside regions thereof with a mixmer design, which comprise sugar modified nucleosides, such as 2′ sugar modified nucleosides, and short regions of DNA nucleosides, such as 1, 2 or 3 DNA nucleosides. Mixmers are exemplified herein by every second design, wherein the nucleosides alternative between 1 LNA and 1 DNA nucleoside, e.g. LDLDLDLDLDLDLDLL, with 5′ and 3′ terminal LNA nucleosides, and every third design, such as LDDLDDLDDLDDLDDL, where every third nucleoside is a LNA nucleoside.

A totalmer is an antisense oligonucleotide or a contiguous nucleotide sequence thereof which does not comprise DNA or RNA nucleosides, and may for example comprise only 2′-O-MOE nucleosides, such as a fully MOE phosphorothioate, e.g. MMMMMMMMMMMMMMMMMMMM, where M=2′-O-MOE, which are reported to be effective splice modulators for therapeutic use. Alternatively, a mixmer may comprise a mixture of modified nucleosides, such as MLMLMLMLMLMLMLMLMLML, wherein L=LNA and M=a non LNA modified nucleoside such as a 2′-O-MOE nucleosides.

Advantageously, the internucleoside nucleosides in mixmers and totalmers may be phosphorothioate, or a majority of nucleoside linkages in mixmers may be phosphorothioate. Mixmers and totalmers may comprise other internucleoside linkages, such as phosphodiester or phosphorodithioate, by way of example.

### Region D′ or D″ in an Oligonucleotide

The contiguous sequence of nucleobases of the oligonucleotide of the invention is typically complementary to multiple TDP-43 binding sites present in distinct TDP-43 RNA targets. The region of the antisense oligonucleotide which is complementary to, such as fully complementary to, the TDP-43 binding site is referred to as the contiguous nucleotide sequence. In some embodiments all of the nucleosides of the antisense oligonucleotide are within the contiguous nucleotide sequence (i.e. the antisense oligonucleotide and contiguous nucleotide sequence are of the same length of nucleotides). In some embodiments the antisense oligonucleotide comprises the contiguous nucleotide sequence and optionally a nucleotide based linker region which may link the oligonucleotide to an optional functional group such as a conjugate, or other non-complementary terminal nucleotides (e.g. region D′ or D″).

The oligonucleotide of the invention may in some embodiments comprise or consist of the contiguous nucleotide sequence of the oligonucleotide which is complementary to the target nucleic acid, such as a mixmer or toalmer region, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein. The addition of region D′ or D″ may be used for the purpose of joining the contiguous nucleotide sequence, such as the mixmer or totoalmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In one embodiment the antisense oligonucleotide of the invention comprises a region D′ and/or D″ in addition to the contiguous nucleotide sequence which constitutes a mixmer or a totalmer. In some embodiments the internucleoside linkage positioned between region D′ or D″ and the mixmer or totalmer region is a phosphodiester linkage.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the antisense oligonucleotide of the invention may optionally, comprise a linker region (second region or region B and/or region Y) which is positioned between the antisense oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages,. Phosphodiester containing biocleavable linkers are described in more detail in WO 2014/076195.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The antisense oligonucleotide of the present invention can be constructed of the following regional elements A—C, A—B—C, A—B—Y—C, A—Y—B—C or A—Y—C. In some embodiments the linker (region Y) is an amino alkyl, such as a C2-C36 amino alkyl group, including, for example C6 to C12 amino alkyl groups. In some embodiments the linker (region Y) is a C6 amino alkyl group.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. In some embodiments, the treatment is not prophylactic for example the treatment is treatment of an existing disease condition which has been diagnosed in the patient.

### The Antisense Oligonucleotides of the Invention

The antisense oligonucleotide of the invention is complementary to an RNA binding site on multiple independent pre-mRNA transcripts, such as to a TDP-43 RNA binding site on multiple pre-mRNA transcripts. The antisense oligonucleotide of the invention is capable or modulating the expression of the multiple pre-mRNA transcripts, for example via (independently) modulation of pre-mRNA splicing, enhancing RNA stabilization, enhancing expression of the encoded protein, reducing expression of truncated proteins encoded by the pre-mRNA(s). As illustrated in the examples, the antisense oligonucleotide of the invention may therefore be used to enhance the fidelity of pre-mRNA processing into mature mRNA encoding correctly expressed and functional proteins. The antisense oligonucleotides of the invention may therefore be suitable for use in the treatment of diseases which are associated with a dysregulation of pre-mRNA maturation.

In some embodiments, the antisense oligonucleotide of the invention may comprise one, two, three, four, five, six, seven, eight or more mismatches between the antisense oligonucleotide and the target nucleic acid TDP-43 binding region. Despite mismatches hybridization to the target nucleic acid may still be sufficient to show a desired modulation of the TDP-43 RNA target RNA. Reduced binding affinity resulting from mismatches may advantageously be compensated by increased number of nucleotides in the antisense oligonucleotide and/or an increased number of modified nucleosides capable of increasing the binding affinity to the target, such as 2′ sugar modified nucleosides, including LNA, present within the oligonucleotide sequence.

In some embodiments, one, two, three, four, five six, seven, eight or more universal nucleosides, such as inosine, may be used at mismatch positions.

Inosine is a nucleoside having the following structure:

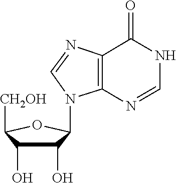

Universal nucleosides are particularly useful when antisense oligonucleotides are targeted to different TDP-43 target RNAs which have non-identical TDP-43 binding regions.

In some embodiments the contiguous nucleotide sequence may include one or more universal nucleotides at positions representing mismatches to a TDP-43 binding site, or target sequence.

In some embodiments the contiguous nucleotide sequence may include two or more universal nucleotides at positions representing mismatches to a TDP-43 binding site, or target sequence.

In some embodiments the contiguous nucleotide sequence may include three or more universal nucleotides at positions representing mismatches to a TDP-43 binding site, or target sequence.

In some embodiments the contiguous nucleotide sequence may include four or more universal nucleotides at positions representing mismatches to a TDP-43 binding site, or target sequence.

In some embodiments the contiguous nucleotide sequence may include five or more universal nucleotides at positions representing mismatches to a TDP-43 binding site, or target sequence.

In some embodiments the contiguous nucleotide sequence may include six or more universal nucleotides at positions representing mismatches to a TDP-43 binding site, or target sequence.

In some embodiments the contiguous nucleotide sequence may include seven or more universal nucleotides at positions representing mismatches to a TDP-43 binding site, or target sequence.

In some embodiments the contiguous nucleotide sequence may include eight or more universal nucleotides at positions representing mismatches to a TDP-43 binding site, or target sequence.

In some embodiments, antisense oligonucleotides of the present invention which contain one more, such as two or more, three or more, four or more, five or more, six or more, seven or more, or eight or more universal nucleotides at positions representing mismatches, may hybridize to a target nucleic acid with estimated ΔG° values below −10 kcal for oligonucleotides that are 10-32 nucleotides in length.

In some embodiments, antisense oligonucleotides of the present invention which contain one more, such as two or more, three or more, four or more, five or more, six or more, seven or more, or eight or more mismatches, may hybridize to a target nucleic acid with estimated ΔG° values below −12 kcal, −15 kcal, −17 kcal, −20 kcal, −30 kcal, −40 kcal, −50 kcal or −60 kcal for oligonucleotides that are 10-32 nucleotides in length.

Calculation of ΔG° values is discussed above.

In some embodiments, the antisense oligonucleotide of the invention or the contiguous nucleotide sequence thereof comprises or consists of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 contiguous nucleotides in length.

In some embodiments, the antisense oligonucleotide or contiguous nucleotide sequence comprises or consists of a sequence selected from the group consisting of sequences SEQ ID NO 1-83 or SEQ ID NO 118-126. It will be understood that the sequence shown in SEQ ID NO 1-83 or SEQ ID NO 118-126 may include modified nucleobases which function as the shown nucleobase in base pairing, for example 5-methyl cytosine may be used in place of methyl cytosine. Inosine may be used as a universal base.

In some embodiments, the antisense oligonucleotide or contiguous nucleotide sequence comprises or consists of 8 to 30 or 8 to 40 nucleotides in length with at least 75%, such as at least 80%, at least 85%, at least 90% identity, at least 95% identity or more than 95% identity to a sequence selected from the group consisting of SEQ ID NO: 1 to 83 or SEQ ID NO: 118-126. In some embodiments the antisense oligonucleotide or contiguous nucleotide sequence comprises or consists of 8 to 30 or 8 to 40 nucleotides in length with 100% identity to a sequence selected from the group consisting of SEQ ID NO: 1 to 83 or SEQ ID NO: 118-126.

It is understood that the contiguous nucleobase sequences (motif sequence) can be modified to for example increase nuclease resistance and/or binding affinity to the target nucleic acid.

The pattern in which the modified nucleosides (such as high affinity modified nucleosides) are incorporated into the oligonucleotide sequence is generally termed oligonucleotide design. The antisense oligonucleotides of the invention are designed with modified nucleosides and DNA nucleosides. Advantageously, high affinity modified nucleosides are used.

In an embodiment, the antisense oligonucleotide, which comprises a contiguous nucleotide sequence comprising one or more 2′-MOE nucleosides, may further comprise at least 1 further or additionally modified nucleoside, such as at least 2, at least 3, at least 4, at least 5, at least 6, at least 7, at least 8, at least 9, at least 10, at least 11, at least 12, at least 13, at least 14, at least 15, at least 16 modified nucleosides, at least 17 modified nucleosides, at least 18 modified nucleosides, at least 19 modified nucleosides, at least 20 modified nucleosides, at least 21 modified nucleosides, at least 22 modified nucleosides, at least 23 modified nucleosides, at least 24 modified nucleosides, at least 25 modified nucleosides, at least 26 modified nucleosides, at least 27 modified nucleosides, at least 28 modified nucleosides, at least 29 modified nucleosides, at least 30 modified nucleosides, at least 31 modified nucleosides, at least 32 modified nucleosides, at least 33 modified nucleosides, at least 34 modified nucleosides, at least 35 modified nucleosides, at least 36 modified nucleosides, at least 37 modified nucleosides, at least 38 modified nucleosides, at least 39 modified nucleosides or more. In an embodiment the antisense oligonucleotide, which comprises a contiguous nucleotide sequence comprising one or more 2′-MOE nucleosides, may further comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described under “modified nucleoside”, “high affinity modified nucleosides”, “sugar modifications”, “2′ sugar modifications” and Locked nucleic acids (LNA)”.

In an embodiment, the antisense oligonucleotide, which comprises a contiguous nucleotide sequence comprising one or more 2′-MOE nucleosides, may further comprise one or more sugar modified nucleosides, such as 2′ sugar modified nucleosides. Preferably the oligonucleotide of the invention may further comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In a further embodiment the antisense oligonucleotide comprises at least one modified internucleoside linkage. Suitable internucleoside modifications are described under “Modified internucleoside linkage”. It is advantageous if at least 75%, such as all, the internucleoside linkages within the contiguous nucleotide sequence are phosphorothioate or boranophosphate internucleoside linkages. In some embodiments all the internucleotide linkages in the contiguous sequence of the antisense oligonucleotide are phosphorothioate linkages.

### Pharmaceutically Acceptable Salts

The term “salts” as used herein conforms to its generally known meaning, i.e. an ionic assembly of anions and cations.

The invention contemplates pharmaceutically acceptable salts of the antisense oligonucleotides of the invention. Put another way, the invention provides for pharmaceutically acceptable salts of the antisense oligonucleotide of the invention.

In some embodiments, the pharmaceutically acceptable salt is a sodium salt, a potassium salt or an ammonium salt.

The invention provides for a pharmaceutically acceptable sodium salt of the antisense oligonucleotide of the invention.

The invention provides for a pharmaceutically acceptable potassium salt of the antisense oligonucleotide of the invention.

The invention provides for a pharmaceutically acceptable ammonium salt of the antisense oligonucleotide of the invention.

### Delivery of Antisense Oligonucleotide Splice Modulators

The invention provides for antisense oligonucleotide of the invention wherein the antisense oligonucleotide are encapsulated in a lipid-based delivery vehicle, covalently linked to or encapsulated in a dendrimer, or conjugated to an aptamer.

This may be for the purpose of delivering the antisense oligonucleotide of the invention to the targeted cells and/or to improve the pharmacokinetics of the antisense oligonucleotide.

Examples of lipid-based delivery vehicles include oil-in-water emulsions, micelles, liposomes, and lipid nanoparticles.

### Method of Manufacture

In a further aspect, the invention provides methods for manufacturing the antisense oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the antisense oligonucleotide. In a further aspect a method is provided for manufacturing the composition of the invention, comprising mixing the antisense oligonucleotide of the invention with a pharmaceutically acceptable diluent, solvent, carrier, salt and/or adjuvant.

### Pharmaceutical Composition

In a further aspect, the invention provides pharmaceutical compositions comprising any of the aforementioned antisense oligonucleotides and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the antisense oligonucleotide is used in the pharmaceutically acceptable diluent at a concentration of 50-300 μM solution.

### Applications

The antisense oligonucleotides of the invention may be utilized as research reagents for, for example, diagnostics, therapeutics and prophylaxis.

In research, such antisense oligonucleotides may be used to mimic the activity of TDP-43 in cells (e.g. in vitro cell cultures, such as in neuronal cells) and experimental animals thereby facilitating functional analysis of the target or an appraisal of its usefulness as a target for therapeutic intervention.

The present invention provides a method, such as an in vivo or in vitro method, for enhancing TDP-43 functionality in a cell which is expressing aberrant or depleted levels of TDP-43, said method comprising administering an antisense oligonucleotide or pharmaceutical composition according to the invention, in an effective amount to said cell. In some embodiments, the target cell, is a mammalian cell in particular a human cell. The target cell may be an in vitro cell culture or an in vivo cell forming part of a tissue in a mammal. In preferred embodiments the target cell is a neuronal cell, such as a neuronal cell which is depleted in normal TDP-43 activity. In some embodiments, the target cell may express a disease associated variant of TDP-43, and/or express dysfunctional TDP-43.

For therapeutics, the antisense oligonucleotides may be administered to an animal or a human, suspected of having a disease or disorder, which can be treated by mimicking TDP-43.

The invention provides methods for treating or preventing a disease, comprising administering a therapeutically or prophylactically effective amount of an antisense oligonucleotide or a pharmaceutical composition of the invention to a subject suffering from or susceptible to the disease.

The invention also relates to an antisense oligonucleotideor a pharmaceutical composition as defined herein for use as a medicament.

The antisense oligonucleotide or a pharmaceutical composition according to the invention is typically administered in an effective amount.

The invention also provides for the use of the antisense oligonucleotide of the invention as described for the manufacture of a medicament for the treatment of a disorder according to the invention, or for a method of the treatment of as a disorder as referred to herein.

The invention further relates to use of an antisense oligonucleotide or a pharmaceutical composition as defined herein for the manufacture of a medicament for the treatment of a neurological disorder as neurodegenerative disorders characterized by TDP-43 pathology or mislocalization of TDP-43 from the nucleus, such as ALS.

The invention also provides the antisense oligonucleotide of the invention for use in a method of treating a disease or disorder as referred to herein.

In one embodiment, the invention relates to antisense oligonucleotides or pharmaceutical compositions for use in the treatment of a neurological disorder as neurodegenerative disorders characterized by TDP-43 pathology or mislocalization of TDP-43 from the nucleus, such as ALS.

The disease or disorder may be selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease,

Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

### Administration

The antisense oligonucleotides or pharmaceutical compositions of the present invention may, for example be administered for example via intracerebral, intracerebroventricular or intrathecal administration.

In a preferred embodiment the antisense oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active antisense oligonucleotide or pharmaceutical composition is administered intravenously. In another embodiment the active antisense oligonucleotide or pharmaceutical composition is administered subcutaneously.

### Combination Therapies

In some embodiments, the antisense oligonucleotide of the invention or pharmaceutical composition of the invention is for use in a combination treatment with one or more other therapeutic agents.

### EXAMPLES

### Example 1: Identification of mRNAs that Becomes Erroneously Spliced in the Absence of TDP43 Protein

One hallmark feature of ALS disease is the presence of cytoplasmic aggregated TDP43 protein in a small fraction of the patient's neuronal cells. The consequence of cytoplasmic aggregation of TDP43 is that it becomes depleted in the cell nucleus, and hence can't perform its normal function here.

TDP43 has been shown to affect mRNA splicing. In order to identify new genes whose mRNAs are regulated by the presence of TDP43, we did a knockdown of TDP43 in a neuronal cell model. RNA sequencing was performed on the cells, and de novo transcript analysis was performed to identify affected genes with new splice patterns.

Human glutamatergic neurons (Fujifilms) were plated at 60,000 viable cells together with 10,000 viable Astrocytes (Fujifilms) in 96-well plates coated with Laminin and Poly(ethyleneimine) solution (Sigma Aldrich) in 200 μl culture medium (day −1).

To knockdown TDP-43, compound A (ID NO: 1, SEQ ID 13) was added to the culture medium at 5 UM on day 0, in other wells PBS was added instead as control. Half the cell culture medium was changed 3 times a week during the whole experiment (day 2, 5, 7, 10, 12, 14 & 17). The cells were harvested on day 20 using Magnapure lysis buffer (Roche) and RNA was isolation on MagNA pure 96 system (Roche) according to the manufacturer's instructions including DNase treatment step. NGS libraries was prepared from 100 ng of total RNA using the KAPA mRNA HyperPrep Kit Illumina® Platforms (Roche). Libraries were subjected to paired-end sequencing on a NovaSeq6000 sequencer (Illumina) with 150-bp read length. Data analysis was carried out using CLC Genomics Workbench 21. Data was first analyzed by running large gap mapping analysis using hg38 genome assembly, followed by transcript discovery. Predicted novel splice events were examined by manual visual inspection to identify real splice events. Here are some of the identified mis-splicing events as a result of TDP43 depletion which we later restored again using an ASO (example 2).

STMN2: Inclusion of a novel exon 2, containing a polyA signal site, resulting in a truncated transcript, producing a truncated STMN2 protein. The first base of the novel exon 2, is positioned at Chr 8 pos 79,616,822 (hg38).

CAMK2B: Novel use of splice doner site resulting in an elongated exon, causing nonsense mediated decay of the transcript. The last base of the elongated exon is located at position Chr 7 pos 44,222,117 (hg38) that is splice together with the next canonical splice acceptor site at Chr 7 pos 44,220,901 (hg38).

KALRN: Inclusion of a novel exon resulting in a pre-mature stop codon and non-sense mediate decay of the transcript. The novel exon had two possible splice acceptor sites and the same splice doner site resulting in an exon with the first and last base being either chr 3 (hg38): (124,700,977; 124,701,255) or (124,701,093-124,701,255).

UNC13A: Inclusion of a novel exon resulting in a pre-mature stop codon and non-sense mediate decay of the transcript. The novel exon had two possible splice acceptor sites and the same splice doner site resulting in an exon with the first and last base being either chr 19 (hg38): (17,642,591; 17,642,414) or (17,642,541-17,642,414). UNC13A is located on the minus strand.

ACTL6B: The inclusion of a novel 69 base pair exon in ACTL6B upon loss of TDP43 was discovered. The first and last base in the new exon is 100,650,643 and 100,650,575 According to the hg38 human gene annotation with the ACTL6B being placed in the minus orientation.

### Example 2: Improved Restoration of Splicing Using a CA-Repeat MOE ASO with Cholesterol Conjugation in TDP43 Depleted Glutaneurons

Here we show CA-repeat ASOs ability to induce proper splicing on the TDP43 targets STMN2, KALRN, CAMK2B, ACTL6B and UNC13A. We show that MOE CA-repeat ASOs with cholesterol conjugated to the 5′ end have an especially potent ability to restore splicing of the affected target genes.

Human glutamatergic neurons (Fujifilms) were plated at 60,000 viable cells together with 10,000 viable Astrocytes (Fujifilms) in 96-well plates coated with Laminin and Poly(ethyleneimine) solution (Sigma Aldrich) in 200 μl culture medium (day −1). To knockdown TDP-43, compound A (ID NO: 1, SEQ ID 13) was added to the culture medium at 5 PM on day 0 (Except for four control wells per plate). Half the cell culture medium was changed 3 times a week during the whole experiment (day 2, 5, 7, 9, 12, 14, 16 & 19). CA-repeat ASOs were added to the cell culture medium on day 5 in variable doses. (25 μM, 7.91 μM, 2.5 μM, 0.791 μM, 0.25 μM, 0.0791 μM, 0.025 μM, 0.00791 μM).

In total the cells were treated with 7 different ASOs containing CA-repeats aswell as one negative control ASO (table 1). 12 wells in the plate received only the compound A (SEQ ID 13) to serve as a baseline reference.

The cells were harvested on day 21 using Magnapure lysis buffer (Roche) and RNA was isolated on MagNA pure 96 system (Roche) according to the manufacturer's instructions including DNase treatment step. The purified RNA was denatured 30 sec at 90 before cDNA synthesis. cDNA was created using the iScript Advanced cDNA Synthesis Kit for RT-qPCR (Biorad) according to the manufacturer's instructions.

Measurements of the expression levels of the target genes was done by droplet digital PCR using the QX1 system (Bio-Rad) together with the QX1 software stand edition. The PCR-probe assays used to measure the expressed of normally spliced target mRNA was designed to span the two exons, where in-between the new “mutant” exon would occur.

The expression value from the experiments are shown in Table 1. The RNA was examined measuring four genes in a run quadruple ddPCR reaction. Mix 1 (TARDBP, STMN2, KALRN, HPRT1) Mix 2 (Custom designed PCR probe assay synthesized at (Integrated DNA technologies (IDT)) or pre-designed ddPCR assay from biorad were used:

Probe mix1
TARDBP:
Primer 1:
CAGCTCATCCTCAGTCATGTC,
Primer 2:
GATGGTGTGACTGCAAACTTC,
Probe:
/5Cy5/CAGCGCCCCACAAACACTTTTCT/3IAbRQSp/)
STMN2:
Primer 1:
CTGCTCAGCGTCTGC,
Primer 2:
GTTGCGAGGTTCCGG,
Probe:
/5HEX/CTAAAACAG/ZEN/CAATGGCCTACAAGGAAAAAATGAAG/
3IABKFQ/
KALRN:
Primer 1:
CGAGCCCTCGGAGTTTG,
Primer 2:
TCCTTCCAAGAAATGGTGGC,
Probe:
/56-FAM/CGACTTCCA/ZEN/GAATATGATGCTGCTGCTGATG/
3IABKFQ/

The following CY5.5 labelled HPRT1 probe was purchased from BioRad: dHsaCPE13136107.

Probe mix 2:
ACTL6B:
Primer 1:
TCTGAGCCAAACCTGCAC,
Primer 2:
ATCAGCTCTGTCAGCTTCTCC,
Probe:
/5HEX/CGAGGCTCC/ZEN/GTGGAACACACG/3IABKFQ/)
UNC13A:
Primer 1:
GATCAAAGGCGAGGAGAAGG,
Primer 2:
TGGCATCTGGGATOTTCAC,
Probe:
/56-FAM/ACCTGTCTG/ZEN/CATGAGAACCTGTTCCACTTC/
3IABKFQ/
CAMK2B:
Primer 1:
CTGACAGTGCCAATACCACC,
Primer 2:
GCTGCTCCGTGGTCTTAAT,
Probe:
/5Cy5/ATGAAGACGCTAAAGCCCGGAAGCAG/3IAbRQSp/

The following CY5.5 labelled GAPDH probe was purchased from BioRad: dHsaCPE70459273.

TABLE 1
Compound Table

| | Natural | | | | |
|---|---|---|---|---|---|
| | analog | Oligo | | | |
| ID NO | sequence | SEQ ID NO | HELM | Natural Target sequence | Target SEQ ID NO |
| ID NO: 1 | TCCACACT | SEQ ID NO 13 | RNA1{[LR](T)[sP].[LR]([5meC])[sP].[dR] | GGTTTGTTCAGTGTGG | SEQ ID NO 96 |
| | GAACAAAC | | (C)[sP].[dR](A)[sP].[dR](C)[sP].[dR](A)[sP] | A | |
| | C | | .[dR](C)[P].[dR](T)[sP].[dR](G)[sP].[dR] | | |
| | | | (A)[sP].[dR](A)[sP].[dR](C)[sP].[dR](A)[sP] | | |
| | | | .[LR](A)[sP].[LR](A)[sP].[LR]([5meC])[sP]. | | |
| | | | [LR]([5meC])}.0 | | |
| ID NO: 2 | CACACACA | SEQ ID NO 21 | RNA1{[LR]([5meC])[sP].[LR](A)[sP].[dR] | GTGTGTGTGTGTGTGT | SEQ ID NO 97 |
| | CACACACA | | (C)[sP].[LR](A)[sP].[dR](C)[sP].[LR](A)[sP] | GTGTGTGTGTGTG | |
| | CACACACA | | .[dR](C)[sP].[LR](A)[P].[dR](C)[sP].[LR] | | |
| | CACAC | | (A)[sP].[dR](C)[sP].[LR](A)[sP].[dR](C)[sP] | | |
| | | | .[LR](A)[sP].[dR](C)[sP].[LR](A)[sP].[dR] | | |
| | | | (C)[sP].[LR](A)[sP].[dR](C)[sP].[LR](A)[sP] | | |
| | | | .[dR](C)[sP].[LR](A)[sP].[dR](C)[sP].[LR] | | |
| | | | (A)[sP].[dR](C)[sP].[LR](A)[sP].[dR](C)[sP] | | |
| | | | .[LR](A)[sP].[LR]([5meC])}.0 | | |
| ID NO: 3 | CACACACA | SEQ ID NO 17 | RNA1{[MOE]([5meC])[sP].[MOE](A)[sP]. | GTGTGTGTGTGTGTGT | SEQ ID NO 98 |
| | CACACACA | | [MOE]([5meC])[sP].[MOE](A)[P].[MOE] | GTGTGTGTG | |
| | CACACACA | | ([5meC])[sP].[MOE](A)[sP].[MOE]([5meC]) | | |
| | C | | [sP].[MOE](A)[sP].[MOE]([5meC])[sP]. | | |
| | | | [MOE](A)[sP].[MOE]([5meC])[sP].[MOE](A) | | |
| | | | [sP].[MOE]([5meC])[sP].[MOE](A)[sP]. | | |
| | | | [MOE]([5meC])[sP].[MOE](A)[sP].[MOE] | | |
| | | | ([5meC])[sP].[MOE](A)[sP].[MOE]([5meC]) | | |
| | | | [sP].[MOE](A)[sP].[MOE]([5meC])[sP]. | | |
| | | | [MOE](A)[sP].[MOE]([5meC])[sP].[MOE](A) | | |
| | | | [sP].[MOE]([5meC])}.0 | | |
| ID NO: 4 | CACACACA | SEQ ID NO 21 | RNA1{[MOE]([5meC])[sP].[MOE](A)[sP]. | GTGTGTGTGTGTGTGT | SEQ ID NO 97 |
| | CACACACA | | [MOE]([5meC])[sP].[MOE](A)[sP].[MOE] | GTGTGTGTGTGTG | |
| | CACACACA | | ([5meC])[sP].[MOE](A)[sP].[MOE]([5meC]) | | |
| | CACAC | | [sP].[MOE](A)[sP].[MOE]([5meC])[sP]. | | |
| | | | [MOE](A)[sP].[MOE]([5meC])[sP].[MOE](A) | | |
| | | | [sP].[MOE]([5meC])[sP].[MOE](A)[sP]. | | |
| | | | [MOE]([5meC])[sP].[MOE](A)[P].[MOE] | | |
| | | | ([5meC])[sP].[MOE](A)[sP].[MOE]([5meC]) | | |
| | | | [sP].[MOE](A)[sP].[MOE]([5meC])[sP]. | | |
| | | | [MOE](A)[sP].[MOE]([5meC])[sP].[MOE](A) | | |
| | | | [sP].[MOE]([5meC])[sP].[MOE](A)[sP]. | | |
| | | | [MOE]([5meC])[sP].[MOE](A)[sP].[MOE]([5 | | |
| | | | meC])}.0 | | |
| ID NO: 5 | [CholTEG]- | SEQ ID NO 17 | CHEM1{[CholTEG]}\|RNA1{[sP].[MOE] | GTGTGTGTGTGTGTGT | SEQ ID NO 98 |
| | CACACACA | | ([5meC])[sP].[MOE](A)[sP].[MOE]([5meC]) | GTGTGTGTG | |
| | CACACACA | | [sP].[MOE](A)[sP].[MOE]([5meC])[sP]. | | |
| | CACACACA | | [MOE](A)[sP].[MOE]([5meC])[sP].[MOE](A) | | |
| | C | | [sP].[MOE]([5meC])[sP].[MOE](A)[P]. | | |
| | | | [MOE]([5meC])[sP].[MOE](A)[sP].[MOE] | | |
| | | | ([5meC])[sP].[MOE](A)[sP].[MOE]([5meC]) | | |
| | | | [sP].[MOE](A)[sP].[MOE]([5meC])[P]. | | |
| | | | [MOE](A)[sP].[MOE]([5meC])[sP].[MOE](A) | | |
| | | | [sP].[MOE]([5meC])[sP].[MOE](A)[sP]. | | |
| | | | [MOE]([5meC])[sP].[MOE](A)[sP].[MOE] | | |
| | | | ([5meC])}$CHEM1,RNA1,1:R1-1:R1.0 | | |
| ID NO: 6 | [CholTEG]- | SEQ ID NO 17 | CHEM1{[CholTEG]}\|RNA1{P.[MOE] | GTGTGTGTGTGTGTGT | SEQ ID NO 98 |
| | CACACACA | | ([5meC])[sP].[MOE](A)[sP].[MOE]([5meC]) | GTGTGTGTG | |
| | CACACACA | | [sP].[MOE](A)[sP].[MOE]([5meC])[sP].[MOE] | | |
| | CACACACA | | (A)[sP].[MOE]([5meC])[sP].[MOE](A)[sP]. | | |
| | C | | [MOE]([5meC])[sP].[MOE](A)[sP].[MOE] | | |
| | | | ([5meC])[sP].[MOE](A)[sP].[MOE]([5meC]) | | |
| | | | [sP].[MOE](A)[P].[MOE]([5meC])[sP]. | | |
| | | | [MOE](A)[sP].[MOE]([5meC])[sP].[MOE](A) | | |
| | | | [sP].[MOE]([5meC])[sP].[MOE](A)[sP]. | | |
| | | | [MOE]([5meC])[sP].[MOE](A)[sP].[MOE] | | |
| | | | ([5meC])[sP].[MOE](A)[sP].[MOE]([5meC])} | | |
| | | | $CHEM1,RNA1,1:R1-1:R1.0 | | |
| ID NO: 7 | [CholTEG]- | SEQ ID NO 21 | CHEM1{[CholTEG]}\|RNA1{[MOE] | GTGTGTGTGTGTGTGT | SEQ ID NO 97 |
| | CACACACA | | ([5meC])[sP].[MOE](A)[sP].[MOE]([5meC])[sP]. | GTGTGTGTGTGTG | |
| | CACACACA | | [MOE](A)[sP].[MOE]([5meC])[sP].[MOE] | | |
| | CACACACA | | (A)[sP].[MOE]([5meC])[sP].[MOE](A)[sP]. | | |
| | CACAC | | [MOE]([5meC])[sP].[MOE](A)[sP].[MOE] | | |
| | | | ([5meC])[sP].[MOE](A)[sP].[MOE]([5meC]) | | |
| | | | [sP].[MOE](A)[sP].[MOE]([5meC])[sP]. | | |
| | | | [MOE](A)[sP].[MOE]([5meC])[sP].[MOE](A) | | |
| | | | [sP].[MOE]([5meC])[sP].[MOE](A)[sP]. | | |
| | | | [MOE]([5meC])[sP].[MOE](A)[sP].[MOE] | | |
| | | | ([5sP].[MOE](A)[sP].[MOE]([5meC])[sP]. | | |
| | | | [MOE](A)[sP].[MOE]([5meC])}\|RNA2{[sP]} | | |
| | | | $RNA2,CHEM1,1:R2-1:R1\|RNA1,RNA2, | | |
| | | | 1:R1-1:R1.0 | | |
| ID NO: 8 | [CholTEG]- | SEQ ID NO 21 | CHEM1{[CholTEG]}\|RNA1{[MOE] | GTGTGTGTGTGTGTGT | SEQ ID NO 97 |
| | CACACACA | | ([5meC])[sP].[MOE](A)[sP].[MOE]([5meC])[sP]. | GTGTGTGTGTGTG | |
| | CACACACA | | [MOE](A)[sP].[MOE]([5meC])[sP].[MOE] | | |
| | CACACACA | | (A)[sP].[MOE]([5meC])[sP].[MOE](A)[sP]. | | |
| | CACAC | | [MOE]([5meC])[sP].[MOE](A)[sP].[MOE] | | |
| | | | ([5meC])[sP].[MOE](A)[sP].[MOE]([5meC]) | | |
| | | | [sP].[MOE](A)[sP].[MOE]([5meC])[sP]. | | |
| | | | [MOE](A)[sP].[MOE]([5meC])[sP].[MOE](A) | | |
| | | | [sP].[MOE]([5meC])[sP].[MOE](A)[sP]. | | |
| | | | [MOE]([5meC])[sP].[MOE](A)[sP].[MOE] | | |
| | | | ([5meC])[sP].[MOE](A)[sP].[MOE]([5meC]) | | |
| | | | [sP].[MOE](A)[sP].[MOE]([5meC])[sP]. | | |
| | | | [MOE](A)[sP].[MOE]([5meC])}\|RNA2{P}$RNA2, | | |
| | | | CHEM1,1:R2-1:R1\|RNA1,RNA2,1:R1-1:R1.0 | | |
| ID NO: 9 | CCAAATCTT | SEQ ID NO 83 | RNA1{[LR]([5meC])[sP].[dR](C)[sP].[LR] | GTAGTTATTATAAGATT | SEQ ID NO 99 |
| | ATAATAACT | | (A)[sP].[LR](A)[sP].[LR](A)[sP].[dR](T)[sP] | TGG | |
| | AC | | .[dR](C)[sP].[dR](T)[sP].[dR](T)[sP].[dR] | | |
| | | | (A)[sP].[dR](T)[P].[dR](A)[sP].[dR](A)[sP] | | |
| | | | .[dR](T)[sP].[dR](A)[sP].[LR](A)[sP].[LR] | | |
| | | | ([5meC])[sP].[dR](T)[sP].[LR](A)[sP].[LR] | | |
| | | | ([5meC])}.0 | | |

Helm Annotation Key:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5-methyl cytosine nucleoside,
[MOE](G) is a 2′-O-methoxyethyl-RNA guanine nucleoside,
[MOE](T) 2′-O-methoxyethyl-RNA thymine nucleoside,
[MOE](A) 2′-O-methoxyethyl-RNA adenine nucleoside,
[MOE]([5meC] 2′-O-methoxyethyl-RNA 5-methyl cytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[CholTEG] is a triethylene glycol linked cholesterol

TABLE 2
Rescue of TDP43 target mRNA splicing following exposure to oligonucleotides

| | ASO rescue | | | | | | |
|---|---|---|---|---|---|---|---|
| | concentration | | | | | | |
| Treatment | uM | TDP43 | STMN2 | KALRN | ACTL6B | UNC13A | CAMK2B |
| KD | | 4.2 | 13.1 | 1.0 | 0.3 | 3.1 | 4.8 |
| KD | | 4.8 | 14.1 | 0.8 | 0.5 | 2.9 | 8.6 |
| KD | | 9.6 | 8.9 | 1.7 | 0.7 | 3.9 | 8.1 |
| KD | | 8.0 | 12.5 | 3.5 | 0.4 | 3.0 | 6.6 |
| KD | | 5.4 | 16.1 | 1.5 | 0.2 | 4.9 | 5.3 |
| KD | | 5.8 | 22.1 | 4.1 | 1.4 | 4.3 | 8.7 |
| KD | | 3.8 | 16.1 | 1.7 | 0.2 | 2.9 | 6.3 |
| KD | | 5.6 | 18.7 | 3.1 | 0.6 | 3.4 | 8.2 |
| KD | | 5.1 | 16.0 | 1.2 | 0.6 | 2.0 | 6.8 |
| KD | | 6.0 | 13.3 | 1.4 | 0.7 | 2.1 | 5.9 |
| KD | | 3.4 | 13.5 | 1.6 | 0.5 | 2.3 | 5.8 |
| KD | | 5.6 | 14.1 | 1.2 | 0.5 | 2.8 | 5.8 |
| PBS | | 92.8 | 107.5 | 97.1 | 93.4 | 93.0 | 96.8 |
| PBS | | 102.9 | 90.4 | 86.3 | 86.0 | 73.7 | 77.2 |
| PBS | | 101.8 | 105.6 | 109.8 | 92.3 | 105.7 | 115.3 |
| PBS | | 102.6 | 96.4 | 106.8 | 128.2 | 127.6 | 110.7 |
| KD + ID NO: 2 | 25 | 3.2 | 46.2 | 28.5 | 15.1 | 6.6 | 18.9 |
| KD + ID NO: 2 | 7.911 | 2.7 | 37.8 | 17.8 | 11.4 | 7.8 | 20.2 |
| KD + ID NO: 2 | 2.5 | 3.4 | 32.0 | 13.3 | 3.9 | 4.1 | 15.1 |
| KD + ID NO: 2 | 0.792 | 6.0 | 26.8 | 10.4 | 3.1 | 4.7 | 14.4 |
| KD + ID NO: 2 | 0.25 | 5.2 | 22.8 | 4.4 | 1.0 | 4.7 | 9.8 |
| KD + ID NO: 2 | 0.079 | 6.9 | 18.5 | 2.4 | 0.7 | 5.1 | 8.3 |
| KD + ID NO: 2 | 0.025 | 7.9 | 14.4 | 1.0 | 0.4 | 2.5 | 6.6 |
| KD + ID NO: 2 | 0.008 | 6.1 | 13.4 | 0.7 | 0.4 | 2.3 | 5.3 |
| KD + ID NO: 3 | 25 | NA | NA | NA | NA | NA | NA |
| KD + ID NO: 3 | 7.911 | 4.6 | 37.5 | 19.8 | 0.6 | 3.5 | 10.2 |
| KD + ID NO: 3 | 2.5 | 4.6 | 22.1 | 8.1 | 0.8 | 3.1 | 8.4 |
| KD + ID NO: 3 | 0.792 | 7.2 | 14.4 | 1.9 | 0.0 | 3.3 | 12.7 |
| KD + ID NO: 3 | 0.25 | 5.9 | 17.8 | 1.1 | 1.2 | 3.5 | 7.6 |
| KD + ID NO: 3 | 0.079 | 4.9 | 16.8 | 1.2 | 0.5 | 3.7 | 5.5 |
| KD + ID NO: 3 | 0.025 | 4.4 | 11.7 | 2.2 | 0.2 | 2.2 | 5.2 |
| KD + ID NO: 3 | 0.008 | 6.5 | 16.8 | 1.2 | 0.3 | 4.5 | 6.4 |
| KD + ID NO: 5 | 25 | 3.3 | 91.2 | 53.3 | 47.6 | 7.8 | 28.4 |
| KD + ID NO: 5 | 7.911 | 3.7 | 72.4 | 39.7 | 22.2 | 4.1 | 16.4 |
| KD + ID NO: 5 | 2.5 | 1.4 | 58.3 | 32.5 | 14.9 | 3.9 | 15.6 |
| KD + ID NO: 5 | 0.792 | 2.2 | 49.3 | 27.6 | 6.9 | 3.2 | 13.7 |
| KD + ID NO: 5 | 0.25 | 2.7 | 35.2 | 14.8 | 4.2 | 4.5 | 13.3 |
| KD + ID NO: 5 | 0.079 | 4.2 | 23.9 | 5.5 | 0.1 | 2.6 | 9.8 |
| KD + ID NO: 5 | 0.025 | 6.5 | 16.0 | 2.0 | 0.2 | 1.3 | 7.7 |
| KD + ID NO: 5 | 0.008 | 5.2 | 18.8 | 2.0 | 0.4 | 3.7 | 6.1 |
| KD + ID NO: 6 | 25 | 2.5 | 104.1 | 74.8 | 41.0 | 10.8 | 30.2 |
| KD + ID NO: 6 | 7.911 | 2.6 | 85.7 | 50.3 | 16.8 | 9.8 | 19.0 |
| KD + ID NO: 6 | 2.5 | 3.1 | 76.0 | 54.6 | 18.8 | 6.4 | 21.3 |
| KD + ID NO: 6 | 0.792 | 2.7 | 73.0 | 45.8 | 13.9 | 4.2 | 18.9 |
| KD + ID NO: 6 | 0.25 | 5.3 | 66.3 | 32.6 | 3.9 | 4.7 | 21.8 |
| KD + ID NO: 6 | 0.079 | 2.9 | 54.7 | 27.5 | 2.7 | 4.1 | 16.2 |
| KD + ID NO: 6 | 0.025 | 6.9 | 26.7 | 13.3 | 0.7 | 2.5 | 10.9 |
| KD + ID NO: 6 | 0.008 | 3.8 | 19.7 | 3.0 | 1.0 | 3.4 | 8.4 |
| KD + ID NO: 4 | 25 | 2.8 | 51.6 | 28.7 | 6.3 | 4.8 | 18.7 |
| KD + ID NO: 4 | 7.911 | 5.4 | 45.7 | 23.5 | 1.5 | 4.1 | 12.8 |
| KD + ID NO: 4 | 2.5 | 5.3 | 27.6 | 8.6 | 0.5 | 5.2 | 9.7 |
| KD + ID NO: 4 | 0.792 | 5.3 | 19.7 | 5.5 | 0.2 | 3.3 | 7.7 |
| KD + ID NO: 4 | 0.25 | 5.7 | 18.8 | 0.8 | 0.9 | 4.3 | 5.6 |
| KD + ID NO: 4 | 0.079 | 6.3 | 17.2 | 0.6 | 0.1 | 4.3 | 7.6 |
| KD + ID NO: 4 | 0.025 | 5.9 | 11.9 | 1.3 | 0.4 | 2.5 | 5.7 |
| KD + ID NO: 4 | 0.008 | 6.8 | 17.9 | 0.5 | 0.4 | 4.3 | 6.4 |
| KD + ID NO: 7 | 25 | 3.1 | 99.9 | 57.3 | 47.5 | 7.8 | 25.0 |
| KD + ID NO: 7 | 7.911 | 4.2 | 80.3 | 44.7 | 25.7 | 5.0 | 19.3 |
| KD + ID NO: 7 | 2.5 | 3.5 | 58.4 | 35.2 | 16.5 | 4.1 | 12.7 |
| KD + ID NO: 7 | 0.792 | 2.4 | 49.7 | 14.7 | 2.7 | 3.8 | 15.2 |
| KD + ID NO: 7 | 0.25 | 3.8 | 36.6 | 14.2 | 6.3 | 3.3 | 7.6 |
| KD + ID NO: 7 | 0.079 | 3.9 | 26.7 | 5.6 | 0.5 | 2.2 | 7.4 |
| KD + ID NO: 7 | 0.025 | 4.0 | 16.9 | 3.1 | 0.1 | 2.5 | 5.9 |
| KD + ID NO: 7 | 0.008 | 3.8 | 17.8 | 3.0 | 1.0 | 3.9 | 8.7 |
| KD + ID NO: 8 | 25 | 3.5 | 91.4 | 58.1 | 32.7 | 8.5 | 25.3 |
| KD + ID NO: 8 | 7.911 | 3.5 | 82.6 | 48.7 | 15.7 | 9.3 | 24.2 |
| KD + ID NO: 8 | 2.5 | 3.0 | 73.0 | 44.2 | 14.2 | 6.1 | 20.7 |
| KD + ID NO: 8 | 0.792 | 2.5 | 72.5 | 47.6 | 14.6 | 5.6 | 21.9 |
| KD + ID NO: 8 | 0.25 | 2.4 | 57.8 | 35.9 | 10.1 | 5.1 | 19.4 |
| KD + ID NO: 8 | 0.079 | 3.2 | 54.2 | 21.0 | 5.4 | 4.1 | 14.1 |
| KD + ID NO: 8 | 0.025 | 7.9 | 25.7 | 8.8 | 0.7 | 3.4 | 4.7 |
| KD + ID NO: 8 | 0.008 | 7.0 | 21.2 | 4.7 | 0.4 | 2.7 | 7.5 |
| KD + ID NO: 9 | 25 | 6.7 | 16.7 | 1.7 | 1.0 | 2.4 | 7.3 |
| KD + ID NO: 9 | 7.911 | 4.2 | 17.3 | 0.5 | 0.7 | 4.1 | 6.1 |
| KD + ID NO: 9 | 2.5 | 6.1 | 17.5 | 3.5 | 0.4 | 4.0 | 5.8 |
| KD + ID NO: 9 | 0.792 | 4.1 | 13.0 | 1.3 | 0.5 | 2.7 | 6.3 |
| KD + ID NO: 9 | 0.25 | 6.6 | 15.5 | 2.0 | 0.6 | 3.4 | 6.6 |
| KD + ID NO: 9 | 0.079 | 5.5 | 11.5 | 0.8 | 0.0 | 2.6 | 10.3 |
| KD + ID NO: 9 | 0.025 | 6.2 | 17.5 | 2.2 | 0.2 | 4.2 | 3.8 |
| KD + ID NO: 9 | 0.008 | 4.3 | 19.0 | 2.4 | 0.4 | 2.7 | 6.7 |

Data shown in Table 2 was normalized to the expression of the house keeping gene present in the given PCR setup (either HPRT1 or GAPDH), and finally normalized to the average expression value of the control wells (PBS) that didn't receive any TDP43 knock-down or CA-repeat ASO. The average expression for all of the given condition is shown in the last column. KD (“knockdown”, describes wells that only received treatment with the gapmer ASO that degrades the TDP43 mRNA)

### Certain References

Salter CG, Beijer D, Hardy H, et al. Truncating SLC5A7 mutations underlie a spectrum of dominant hereditary motor neuropathies. Neurol Genet. 2018; 4 (2): e222.

Yukiko Nasu-Nishimura 1, Tomoatsu Hayashi, Tomohiro Ohishi, Toshio Okabe, Susumu Ohwada, Yoshimi Hasegawa, Takao Senda, Chikashi Toyoshima, Tsutomu Nakamura, Tetsu Akiyama. Role of the Rho GTPase-activating Protein RICS in Neurite Outgrowth. Genes Cells. 2006 June;11 (6): 607-14.

Arundhati Jana, Edward L. Hogan, and Kalipada Pahan. Ceramide and neurodegeneration: Susceptibility of neurons and oligodendrocytes to cell damage and death. J Neurol Sci. 2009 Mar. 15; 278 (1-2): 5-15.

Conti et al. TDP-43 affects splicing profiles and isoform production of genes involved in the apoptotic and mitotic cellular pathways. Nucleic Acids Res. 2015 Oct. 15; 43 (18): 8990-9005. Humphrey et al. Quantitative analysis of cryptic splicing associated with TDP-43 depletion. BMC Medical Genomics 2017; volume 10, Article number: 38 (2017).

Melamed et al. Premature polyadenylation-mediated loss of stathmin −2 is a hallmark of TDP-43-dependent neurodegeneration. Nat Neurosci. 2019 February; 22 (2): 180-190.

Klim et al., ALS-implicated protein TDP-43 sustains levels of STMN2, a mediator of motor neuron growth and repair. Nature Neuroscience 22, pages167-179 (2019)

SEQUENCES

| SEQ ID | |
|---|---|
| NO | Sequence 5′-3′ |
| N/A | ACACACAC |
| N/A | ACACACACA |
| 1 | ACACACACAC |
| 2 | ACACACACACA |
| 3 | ACACACACACAC |
| 4 | ACACACACACACA |
| 5 | ACACACACACACACAC |
| 6 | ACACACACACACACACAC |
| N/A | CACACAC |
| N/A | CACACACA |
| N/A | CACACACAC |
| 7 | CACACACACA |
| 8 | CACACACACAC |
| 9 | CACACACACACA |
| 10 | CACACACACACAC |
| 11 | CACACACACACACACA |
| 12 | CACACACACACACACACA |
| 13 | TCCACACTGAACAAACC |
| 14 | CACACACACACACACACACACAC |
| 15 | ACACACACACACACACACACACAC |
| 16 | CACACACACACAACACACACACAC |
| 17 | CACACACACACACACACACACACAC |
| 18 | ACACACACACACACACACACACACAC |
| 19 | CACACACACACACACACACACACACAC |
| 20 | ACACACACACACACACACACACACACAC |
| 21 | CACACACACACACACACACACACACACAC |
| 22 | ACACACACACACACACACACACACACACAC |
| 23 | CACACACACACACACACACACACACACACAC |
| 24 | ACACACACACACACACACCACACACACACACACA |
| 25 | CACACACACACACACACACACACACACACACACA |
| 26 | CACACACACACACACACAACACACACACACACACAC |
| 27 | ACACACACACACACACACCACACACACACACACACA |
| 28 | ACACACACACACACACACACACACACACACAC |
| 29 | CACACACCCACICACACACGCAC |
| 30 | CACACICACACACACACACACAC |
| 31 | CACACACACACICACACACACAC |
| 32 | CACACICACACACACACACACTC |
| 33 | CACATACACACCCACACACACAC |
| 34 | CACACACACACACACACGCACAC |
| 35 | CACICACICACACACICACICAC |
| 36 | CACACACACACACACTCTCACAC |
| 37 | CTCACACACACACACACACACAC |
| 38 | CACACACACACAAACACACACAC |
| 39 | CACACGCACGCACACACACACAC |
| 40 | CACGCACACACCCACACACTCAC |
| 41 | CACACACACACACTCTCTCACAC |
| 42 | CACACACACACACICICICICAC |
| 43 | CACTCACACACTCACACACACAC |
| 44 | CACGCACACACGCACACACACAC |
| 45 | CACCCACACACCCACACACACAC |
| 46 | CACACGCACATACACACACCCAC |
| 47 | TTCACACACACACACACACACAC |
| 48 | CACACACACACACACACACACAA |
| 49 | CACACACACACACACACACAIACACACAC |
| 50 | CACAIACACACACACACACACACACACAC |
| 51 | CATACACACACACACACACATACACACAC |
| 52 | CAAACACACACACACACACACACACACAC |
| 53 | CACACACACTCACACACACACACACACAC |
| 54 | CACAGACACACACACACACACACACACAC |
| 55 | CACAAACACACACACACACACACACACAC |
| 56 | CACATACACACACACACACACACACACAC |
| 57 | CATACGCACATACACGCACACACAAACAC |
| 58 | CACATATACACATACACACACACACACAC |
| 59 | CACACACACACACACACACTCTCTCTCTC |
| 60 | CACGCACACACACACACACACACACACAC |
| 61 | CACACACACACACACAITITITCACACAC |
| 62 | CGCACACACACACACACACACACACACAC |
| 63 | CACACACACACACACAGACAGACAGACAC |
| 64 | CACACACACAAAAAAACACACACACACAC |
| 65 | CACACACACACCCCCCCACACACACACAC |
| 66 | CACACACACTCTCTCACACACACAC |
| 67 | CACAAACACACACACACACACACAC |
| 68 | CACTCACACACACACACACACACAC |
| 69 | CACACACACAAACACACACACACAC |
| 70 | CACCCACACACACACACACACACAC |
| 71 | CTCTCTCACACACACACACACACAC |
| 72 | CAAACACACACACACACACACACAC |
| 73 | CACACACACCCACACACACACACAC |
| 74 | CACACACACACACACCCACACACAC |
| 75 | CACACACACACACACGCACACACAC |
| 76 | CACACACACACACACTCACACACAC |
| 77 | CACACACACATACACACACACACAC |
| 78 | CACACACACAGACACACACACACAC |
| 79 | TTTACACACACACACACACACACAC |
| 80 | CACGCACACGCACACACACACACAC |
| 81 | CACGCACACACGCACACACACGCAC |
| 82 | CACACGCACACACACACACACACAC |
| 83 | CCAAATCTTATAATAACTAC |
| 84 | UGUGUGUGUGU |
| 85 | UGUGUGUGUGUG |
| 86 | UGUGUGUGUGUGU |
| 87 | GUGUGUGUGU |
| 88 | GUGUGUGUGUG |
| 89 | GUGUGUGUGUGU |
| 90 | GUGUGUGUGUGUG |
| 91 | UGUGUGUGUGUGUG |
| 92 | GUGUGUGUGUGUGU |
| 93 | UGUGUGUGUGUGUGUGUG |
| 94 | GUGUGUGUGUGUGUGUGU |
| 95 | UGUGUGUGUG |
| 96 | GGTTTGTTCAGTGTGGA |
| 97 | GTGTGTGTGTGTGTGTGTGTGTGTGTGTG |
| 98 | GTGTGTGTGTGTGTGTGTGTGTGTG |
| 99 | GTAGTTATTATAAGATTTGG |

1. An antisense oligonucleotide of 8 to 40 nucleotides in length, which comprises a contiguous nucleotide sequence of at least 8 nucleotides in length which is complementary to a sequence comprising (5′-3′) (UG) n, (GU) n, wherein n is 4-20, UGUGUGUG, UGUGUGUGU, UGUGUGUGUG (SEQ ID NO 95), UGUGUGUGUGU (SEQ ID NO 84), UGUGUGUGUGUG (SEQ ID NO 85), UGUGUGUGUGUGU (SEQ ID NO 86), GUGUGUGU, GUGUGUGUG, GUGUGUGUGU (SEQ ID NO 87), GUGUGUGUGUG (SEQ ID NO 88), GUGUGUGUGUGU (SEQ ID NO 89), GUGUGUGUGUGUG (SEQ ID NO 90), or GUGAAUGA, wherein the contiguous nucleotide sequence comprises one or more 2′-O-methoxyethyl-RNA (2′-MOE) nucleosides and wherein the antisense oligonucleotide is attached to at least one cholesterol moiety.

2. The antisense oligonucleotide according to claim 1, wherein:
(a) the contiguous nucleotide sequence comprises:
(i) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32, 33, 34, 35, 36, 37, 38, 39, or 40 2′-O-methoxyethyl-RNA (2′-MOE) nucleosides; and/or
(ii) at least 10%, at least 20%, at least 30%, at least 40%, at least 50%, at least 60%, at least 70%, at least 80%, at least 90% or 100% 2′-O-methoxyethyl-RNA (2′-MOE) nucleosides:
(b) the antisense oligonucleotide is attached to two or more or three or more cholesterol moieties;
(c) the at least one cholesterol moiety is selected from the group comprising: 5′-cholesterol-TEG-CE phosphoramidite, 5′-cholesterol-CE phosphoramidite or cholesteryl-TEG-CE phosphoramidite; and/or
(d) a linker is positioned between the antisense oligonucleotide and the at least one cholesterol moiety.

3. (canceled)

4. The antisense oligonucleotide according to claim 1, wherein all of the nucleosides of the contiguous nucleotide sequence are 2′-O-methoxyethyl-RNA (2′-MOE) nucleosides and/or all the internucleoside linkages present in the antisense oligonucleotide are phosphorothioate internucleoside linkages.

5. (canceled)

6. The antisense oligonucleotide according to claim 2, wherein:
(a) the at least one cholesterol moiety is covalently attached to the antisense oligonucleotide; and/or
(b) a linker is positioned between the antisense oligonucleotide and the cholesterol moiety, and wherein the linker is a C3 alkyl group, C6 alkyl group, a C12 alkyl group, a TEG group or a HEG group and/or the linker is a physiologically labile linker.

7-10. (canceled)

11. The antisense oligonucleotide according to claim 6, wherein the linker is a physiologically labile linker, and wherein the physiologically labile linker is a S1 nuclease susceptible linker.

12. The antisense oligonucleotide according to claim 1, wherein the antisense oligonucleotide has the structure shown as:

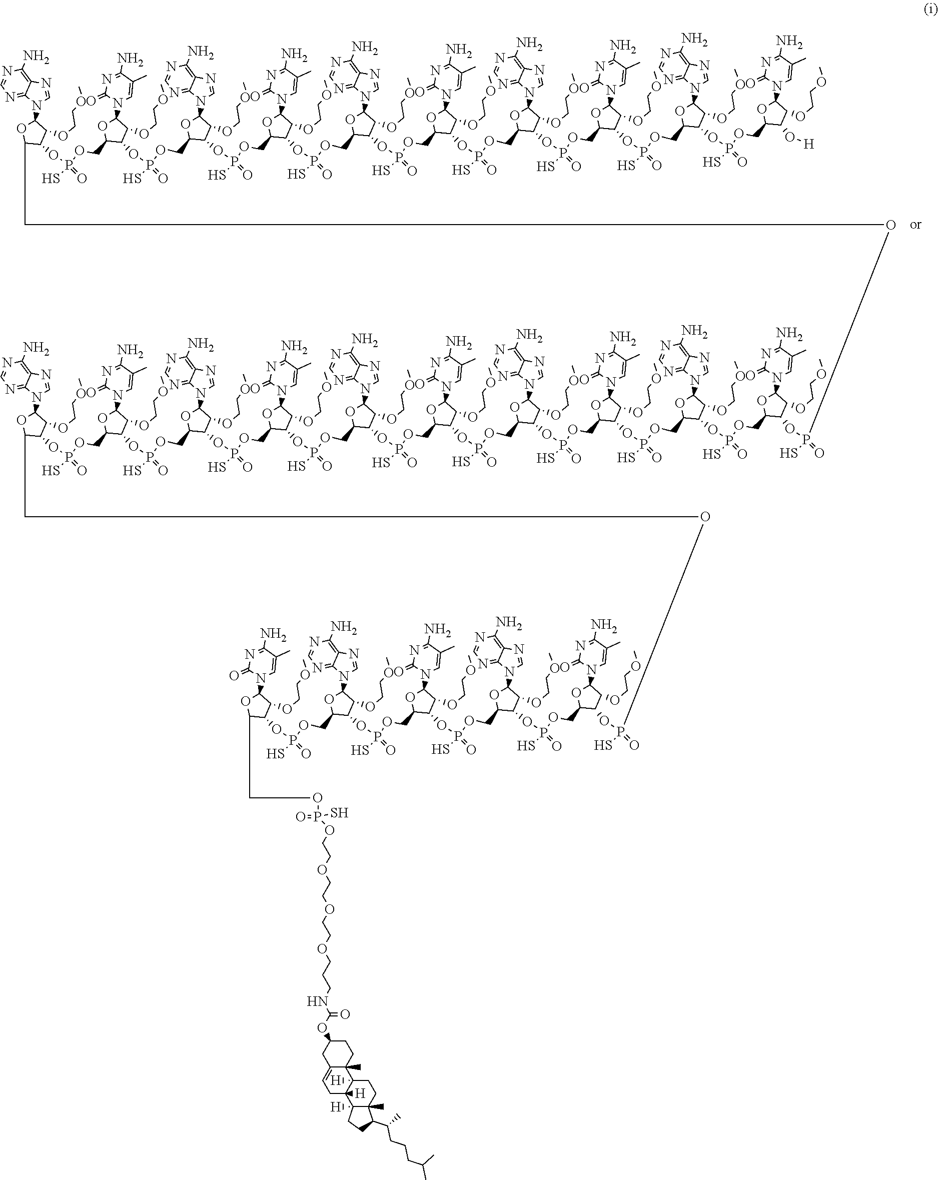

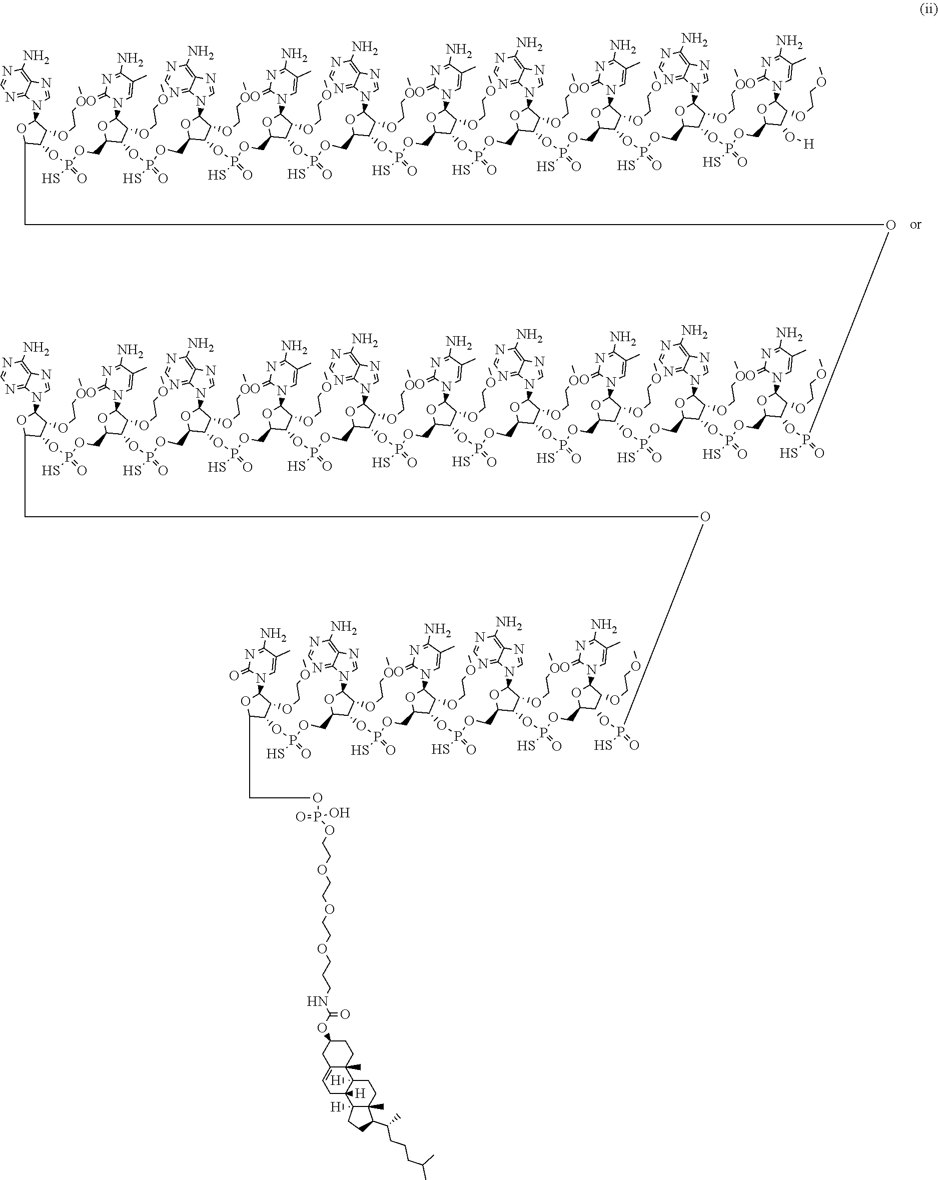

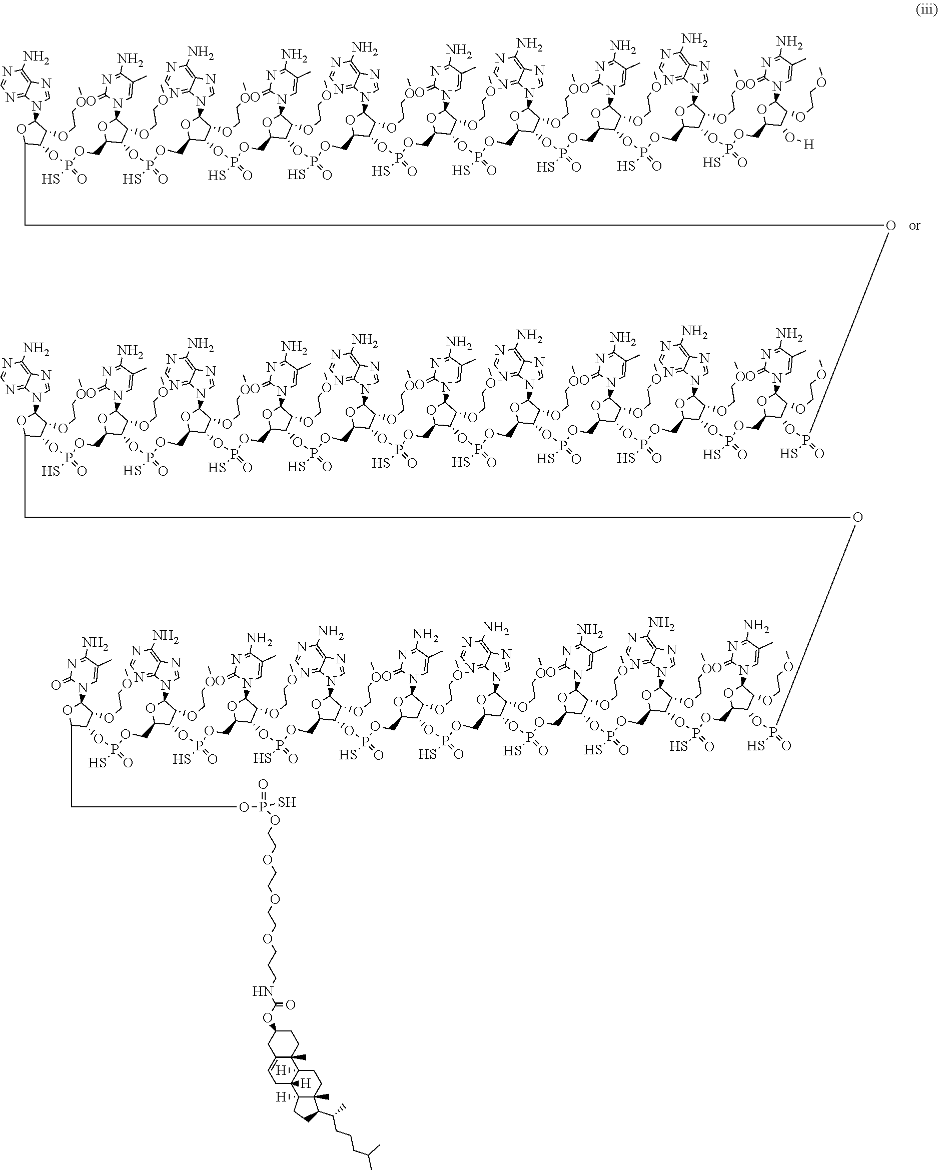

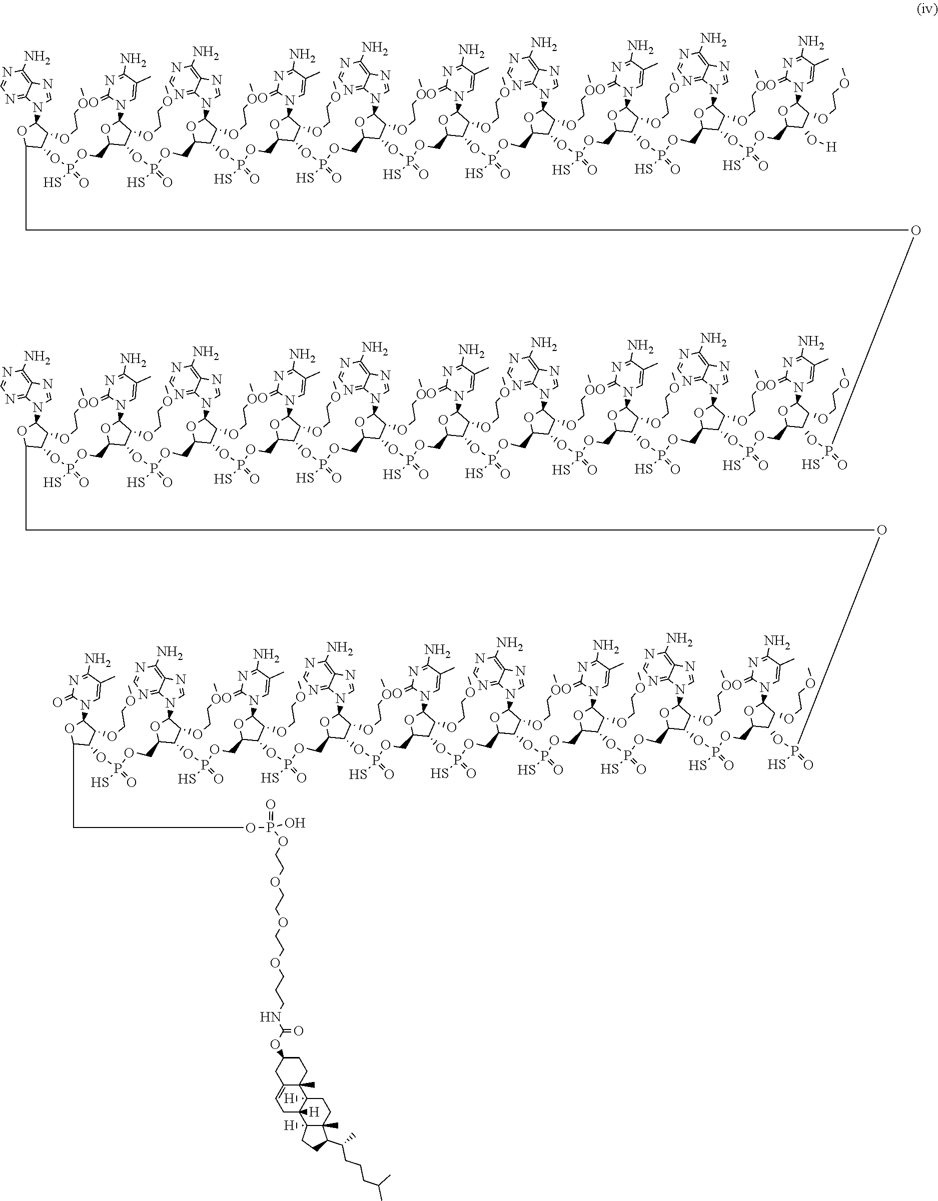

13. The antisense oligonucleotide according to claim 1, wherein the contiguous nucleotide sequence comprises (i) a sequence selected from CACACAC, CACACACA, CACACACAC, ACACACAC, or ACACACACA; or (ii) a sequence selected from the group consisting of comprising any one of SEQ ID No 1-83, or a fragment of 8 or more contiguous nucleotides thereof.

14. (canceled)

15. The antisense oligonucleotide according to claim 1, wherein:
(a) the contiguous nucleotide sequence is at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length;
(b) the antisense oligonucleotide consists of the contiguous nucleotide sequence;
(c) the contiguous nucleotide sequence is at least 75%, at least 80%, at least 85%, at least 90%, or at least 95% complementary to the target sequence; and/or
(d) the contiguous nucleotide sequence comprises 1, 2, 3, 4, 5, 6, 7, 8, or more mismatches to the target sequence.

16-19. (canceled)

20. The antisense oligonucleotide according to claim 1, wherein:
(a) the Gibbs free energy of the antisense oligonucleotide to a complementary target RNA is lower than about −10AG, such as lower than about −15 AG, such as lower than about −17 AG:
(b) the antisense oligonucleotide is capable of restoring the functional phenotype of one or more TDP-43 target RNA(s) in a cell which is TDP-43 depleted, or is expressing aberrant TDP-43 protein;
(c) the antisense oligonucleotide is capable of modulating the splicing of two or more TDP-43 target pre-mRNAs;
(d) the antisense oligonucleotide is capable of enhancing the fidelity of pre-mRNA splicing of two or more mRNAs comprising STMN2 mRNA, CAMK2B mRNA, KALRN mRNA, ACTL6B mRNA or UNC13A mRNA, when administered to a TDP-43 depleted cell:
(e) the antisense oligonucleotide is capable of decreasing the proportion of STMN2 mature mRNA which comprises a cryptic exon (ce1) between exon 1 and exon 2, as compared to the wild-type STMN2 mature mRNA with a contiguous exon1/exon2 junction, when administered to a TDP-43 depleted cell which is expressing STMN2 pre-mRNA;
(f) the antisense oligonucleotide is capable of decreasing the level of aberrant exon inclusion in CAMK2B mRNA transcript, when administered to a TDP-43 depleted cell which is expressing CAMK2B pre-mRNA;
(g) the antisense oligonucleotide is capable of decreasing the level of aberrant exon inclusion in KALRN mRNA transcript, when administered to a TDP-43 depleted cell which is expressing KALRN pre-mRNA;
(h) the antisense oligonucleotide is capable of decreasing the level of aberrant exon inclusion in UNC13A mRNA transcript, when administered to a TDP-43 depleted cell which is expressing UNC13A pre-mRNA;
(i) the antisense oligonucleotide is capable of decreasing the level of aberrant exon inclusion in ACTL6B mRNA transcript, when administered to a TDP-43 depleted cell which is expressing ACTL6B pre-mRNA; and/or
(j) the antisense oligonucleotide is capable of correcting the aberrant splicing of two or more of STMN2, CAMK2B, KALRN, ACTL6B and UNC13A pre-mRNA in a TDP-43 depleted cell.

21-22. (canceled)

23. The antisense oligonucleotide according to claim 20, wherein the two or more TDP-43 target pre-mRNAs comprise are independently selected from the group consisting of STMN2 pre-mRNA, CAMK2B pre-mRNA, KALRN pre-mRNA, ACTL6B pre-mRNA, or UNC13A pre-mRNA.

24-30. (canceled)

31. The antisense oligonucleotide according to claim 1, wherein the antisense oligonucleotide does not comprise a region of more than 3, or more than 4, contiguous DNA nucleosides.

32. The antisense oligonucleotide according to claim 1, wherein the antisense oligonucleotide is not capable of mediating RNAseH cleavage.

33. The antisense oligonucleotide according to claim 1, wherein:
(a) the antisense oligonucleotide is a morpholino antisense oligonucleotide;
(b) the antisense oligonucleotide further comprises one or more modified nucleosides, such as a 2′ sugar modified nucleoside comprising 2′-O-alkyl-RNA; 2′-O-methyl RNA (2′-OMe); 2′-alkoxy-RNA; 2′-amino-DNA; 2′-fluro-RNA; 2′-fluoro-DNA; arabino nucleic acid (ANA): 2′-fluoro-ANA: locked nucleic acid (LNA), or a combination thereof; and/or
(c) one or more of the internucleoside linkages positioned between the nucleosides on the contiguous nucleotide sequence are modified.

34. (canceled)

35. The antisense oligonucleotide according to claim 33, wherein:
(a) the 2′ sugar modified nucleoside is an affinity enhancing 2′ sugar modified nucleoside;
(b) one or more of the modified nucleosides is a locked nucleic acid nucleoside (LNA), such as an LNA nucleoside comprising constrained ethyl nucleoside (cEt) or B-D-oxy-LNA;
(c) the contiguous nucleotide sequence of the antisense oligonucleotide comprises LNA nucleosides and DNA nucleosides; and/or
(d) the antisense oligonucleotide or contiguous nucleotide sequence thereof is a mixmer or a totalmer.

36-39. (canceled)

40. The antisense oligonucleotide according to claim 33, wherein:
(a) at least about 75%, at least about 80%, at least about 85%, at least about 90%, at least about 95%, or about 100% of the internucleoside linkages positioned between the nucleosides on the contiguous nucleotide sequence are modified; and/or
(b) one or more, or all, of the modified internucleoside linkages comprise a phosphorothioate linkage.

41-42. (canceled)

43. The antisense oligonucleotide according to claim 1, wherein the antisense oligonucleotide is covalently attached to at least one conjugate moiety.

44. The antisense oligonucleotide according to claim 1, wherein the antisense oligonucleotide is in the form of a pharmaceutically acceptable salt.

45. The antisense oligonucleotide according to claim 44, wherein the salt is a sodium salt or a potassium salt.

46. A pharmaceutical composition comprising the antisense oligonucleotide according to claim 1 and a pharmaceutically acceptable diluent, solvent, carrier, salt, and/or adjuvant.

47. A method for enhancing TDP-43 functionality in a cell which is expressing aberrant or depleted levels of TDP-43, said method comprising administering the antisense oligonucleotide according to claim 1 in an effective amount to said cell.

48. A method for treating or preventing a TDP-43 pathology in a subject comprising administering a therapeutically or prophylactically effective amount of the antisense oligonucleotide according to claim 1, according to claim 46, to a subject suffering from or susceptible to the TDP-43 pathology.

49-51. (canceled)

52. The method according to claim 48, the antisense oligonucleotide or the pharmaceutical composition for use according to claim 50 or the use according to claim 51, wherein the TDP-43 pathology is a neurological disorder comprising selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, or a polyglutamine disease, such as spinocerebellar ataxia 3, myopathies, and Chronic Traumatic Encephalopathy.

53. The method according to claim 52, wherein the TDP-43 pathology is a neurological disorder comprising amyotrophic lateral sclerosis (ALS) or frontotemporal lobar degeneration (FTLD).


SEQUENCE LISTING (126 sequences)

SEQ ID NO: 1 - DNA, 10 bp, synthetic construct
  source 1..10: mol_type = other DNA
acacacacac

SEQ ID NO: 2 - DNA, 11 bp, synthetic construct
  source 1..11: mol_type = other DNA
acacacacaca

SEQ ID NO: 3 - DNA, 12 bp, synthetic construct
  source 1..12: mol_type = other DNA
acacacacacac

SEQ ID NO: 4 - DNA, 13 bp, synthetic construct
  source 1..13: mol_type = other DNA
acacacacacaca

SEQ ID NO: 5 - DNA, 16 bp, synthetic construct
  source 1..16: mol_type = other DNA
acacacacacacacac

SEQ ID NO: 6 - DNA, 18 bp, synthetic construct
  source 1..18: mol_type = other DNA
acacacacacacacacac

SEQ ID NO: 7 - DNA, 10 bp, synthetic construct
  source 1..10: mol_type = other DNA
cacacacaca

SEQ ID NO: 8 - DNA, 11 bp, synthetic construct
  source 1..11: mol_type = other DNA
cacacacacac

SEQ ID NO: 9 - DNA, 12 bp, synthetic construct
  source 1..12: mol_type = other DNA
cacacacacaca

SEQ ID NO: 10 - DNA, 13 bp, synthetic construct
  source 1..13: mol_type = other DNA
cacacacacacac

SEQ ID NO: 11 - DNA, 16 bp, synthetic construct
  source 1..16: mol_type = other DNA
cacacacacacacaca

SEQ ID NO: 12 - DNA, 18 bp, synthetic construct
  source 1..18: mol_type = other DNA
cacacacacacacacaca

SEQ ID NO: 13 - DNA, 17 bp, synthetic construct
  source 1..17: mol_type = other DNA
tccacactgaacaaacc

SEQ ID NO: 14 - DNA, 23 bp, synthetic construct
  source 1..23: mol_type = other DNA
cacacacacacacacacacacac

SEQ ID NO: 15 - DNA, 24 bp, synthetic construct
  source 1..24: mol_type = other DNA
acacacacacacacacacacacac

SEQ ID NO: 16 - DNA, 24 bp, synthetic construct
  source 1..24: mol_type = other DNA
cacacacacacaacacacacacac

SEQ ID NO: 17 - DNA, 25 bp, synthetic construct
  source 1..25: mol_type = other DNA
cacacacacacacacacacacacac

SEQ ID NO: 18 - DNA, 26 bp, synthetic construct
  source 1..26: mol_type = other DNA
acacacacacacacacacacacacac

SEQ ID NO: 19 - DNA, 27 bp, synthetic construct
  source 1..27: mol_type = other DNA
cacacacacacacacacacacacacac

SEQ ID NO: 20 - DNA, 28 bp, synthetic construct
  source 1..28: mol_type = other DNA
acacacacacacacacacacacacacac

SEQ ID NO: 21 - DNA, 29 bp, synthetic construct
  source 1..29: mol_type = other DNA
cacacacacacacacacacacacacacac

SEQ ID NO: 22 - DNA, 30 bp, synthetic construct
  source 1..30: mol_type = other DNA
acacacacacacacacacacacacacacac

SEQ ID NO: 23 - DNA, 31 bp, synthetic construct
  source 1..31: mol_type = other DNA
cacacacacacacacacacacacacacacac

SEQ ID NO: 24 - DNA, 34 bp, synthetic construct
  source 1..34: mol_type = other DNA
acacacacacacacacaccacacacacacacaca

SEQ ID NO: 25 - DNA, 34 bp, synthetic construct
  source 1..34: mol_type = other DNA
cacacacacacacacacacacacacacacacaca

SEQ ID NO: 26 - DNA, 36 bp, synthetic construct
  source 1..36: mol_type = other DNA
cacacacacacacacacaacacacacacacacacac

SEQ ID NO: 27 - DNA, 36 bp, synthetic construct
  source 1..36: mol_type = other DNA
acacacacacacacacaccacacacacacacacaca

SEQ ID NO: 28 - DNA, 32 bp, synthetic construct
  source 1..32: mol_type = other DNA
acacacacacacacacacacacacacacacac

SEQ ID NO: 29 - DNA, 23 bp, synthetic construct
  source 1..23: mol_type = other DNA
  modified_base 12: mod_base = i
cacacacccacncacacacgcac

SEQ ID NO: 30 - DNA, 23 bp, synthetic construct
  source 1..23: mol_type = other DNA
  modified_base 6: mod_base = i
cacacncacacacacacacacac

SEQ ID NO: 31 - DNA, 23 bp, synthetic construct
  source 1..23: mol_type = other DNA
  modified_base 12: mod_base = i
cacacacacacncacacacacac

SEQ ID NO: 32 - DNA, 23 bp, synthetic construct
  source 1..23: mol_type = other DNA
  modified_base 6: mod_base = i
cacacncacacacacacacactc

SEQ ID NO: 33 - DNA, 23 bp, synthetic construct
  source 1..23: mol_type = other DNA
cacatacacacccacacacacac

SEQ ID NO: 34 - DNA, 23 bp, synthetic construct
  source 1..23: mol_type = other DNA
cacacacacacacacacgcacac

SEQ ID NO: 35 - DNA, 23 bp, synthetic construct
  source 1..23: mol_type = other DNA
  modified_base 4: mod_base = i
  modified_base 8: mod_base = i
  modified_base 16: mod_base = i
  modified_base 20: mod_base = i
cacncacncacacacncacncac

SEQ ID NO: 36 - DNA, 23 bp, synthetic construct
  source 1..23: mol_type = other DNA
cacacacacacacactctcacac

SEQ ID NO: 37 - DNA, 23 bp, synthetic construct
  source 1..23: mol_type = other DNA
ctcacacacacacacacacacac

SEQ ID NO: 38 - DNA, 23 bp, synthetic construct
  source 1..23: mol_type = other DNA
cacacacacacaaacacacacac

SEQ ID NO: 39 - DNA, 23 bp, synthetic construct
  source 1..23: mol_type = other DNA
cacacgcacgcacacacacacac

SEQ ID NO: 40 - DNA, 23 bp, synthetic construct
  source 1..23: mol_type = other DNA
cacgcacacacccacacactcac

SEQ ID NO: 41 - DNA, 23 bp, synthetic construct
  source 1..23: mol_type = other DNA
cacacacacacactctctcacac

SEQ ID NO: 42 - DNA, 23 bp, synthetic construct
  source 1..23: mol_type = other DNA
  modified_base 14: mod_base = i
  modified_base 16: mod_base = i
  modified_base 18: mod_base = i
  modified_base 20: mod_base = i
cacacacacacacncncncncac

SEQ ID NO: 43 - DNA, 23 bp, synthetic construct
  source 1..23: mol_type = other DNA
cactcacacactcacacacacac

SEQ ID NO: 44 - DNA, 23 bp, synthetic construct
  source 1..23: mol_type = other DNA
cacgcacacacgcacacacacac

SEQ ID NO: 45 - DNA, 23 bp, synthetic construct
  source 1..23: mol_type = other DNA
cacccacacacccacacacacac

SEQ ID NO: 46 - DNA, 23 bp, synthetic construct
  source 1..23: mol_type = other DNA
cacacgcacatacacacacccac

SEQ ID NO: 47 - DNA, 23 bp, synthetic construct
  source 1..23: mol_type = other DNA
ttcacacacacacacacacacac

SEQ ID NO: 48 - DNA, 23 bp, synthetic construct
  source 1..23: mol_type = other DNA
cacacacacacacacacacacaa

SEQ ID NO: 49 - DNA, 29 bp, synthetic construct
  source 1..29: mol_type = other DNA
  modified_base 21: mod_base = i
cacacacacacacacacacanacacacac

SEQ ID NO: 50 - DNA, 29 bp, synthetic construct
  source 1..29: mol_type = other DNA
  modified_base 5: mod_base = i
cacanacacacacacacacacacacacac

SEQ ID NO: 51 - DNA, 29 bp, synthetic construct
  source 1..29: mol_type = other DNA
catacacacacacacacacatacacacac

SEQ ID NO: 52 - DNA, 29 bp, synthetic construct
  source 1..29: mol_type = other DNA
caaacacacacacacacacacacacacac

SEQ ID NO: 53 - DNA, 29 bp, synthetic construct
  source 1..29: mol_type = other DNA
cacacacactcacacacacacacacacac

SEQ ID NO: 54 - DNA, 29 bp, synthetic construct
  source 1..29: mol_type = other DNA
cacagacacacacacacacacacacacac

SEQ ID NO: 55 - DNA, 29 bp, synthetic construct
  source 1..29: mol_type = other DNA
cacaaacacacacacacacacacacacac

SEQ ID NO: 56 - DNA, 29 bp, synthetic construct
  source 1..29: mol_type = other DNA
cacatacacacacacacacacacacacac

SEQ ID NO: 57 - DNA, 29 bp, synthetic construct
  source 1..29: mol_type = other DNA
catacgcacatacacgcacacacaaacac

SEQ ID NO: 58 - DNA, 29 bp, synthetic construct
  source 1..29: mol_type = other DNA
cacatatacacatacacacacacacacac

SEQ ID NO: 59 - DNA, 29 bp, synthetic construct
  source 1..29: mol_type = other DNA
cacacacacacacacacactctctctctc

SEQ ID NO: 60 - DNA, 29 bp, synthetic construct
  source 1..29: mol_type = other DNA
cacgcacacacacacacacacacacacac

SEQ ID NO: 61 - DNA, 29 bp, synthetic construct
  source 1..29: mol_type = other DNA
  modified_base 17: mod_base = i
  modified_base 19: mod_base = i
  modified_base 21: mod_base = i
cacacacacacacacantntntcacacac

SEQ ID NO: 62 - DNA, 29 bp, synthetic construct
  source 1..29: mol_type = other DNA
cgcacacacacacacacacacacacacac

SEQ ID NO: 63 - DNA, 29 bp, synthetic construct
  source 1..29: mol_type = other DNA
cacacacacacacacagacagacagacac

SEQ ID NO: 64 - DNA, 29 bp, synthetic construct
  source 1..29: mol_type = other DNA
cacacacacaaaaaaacacacacacacac

SEQ ID NO: 65 - DNA, 29 bp, synthetic construct
  source 1..29: mol_type = other DNA
cacacacacacccccccacacacacacac

SEQ ID NO: 66 - DNA, 25 bp, synthetic construct
  source 1..25: mol_type = other DNA
cacacacactctctcacacacacac

SEQ ID NO: 67 - DNA, 25 bp, synthetic construct
  source 1..25: mol_type = other DNA
cacaaacacacacacacacacacac

SEQ ID NO: 68 - DNA, 25 bp, synthetic construct
  source 1..25: mol_type = other DNA
cactcacacacacacacacacacac

SEQ ID NO: 69 - DNA, 25 bp, synthetic construct
  source 1..25: mol_type = other DNA
cacacacacaaacacacacacacac

SEQ ID NO: 70 - DNA, 25 bp, synthetic construct
  source 1..25: mol_type = other DNA
cacccacacacacacacacacacac

SEQ ID NO: 71 - DNA, 25 bp, synthetic construct
  source 1..25: mol_type = other DNA
ctctctcacacacacacacacacac

SEQ ID NO: 72 - DNA, 25 bp, synthetic construct
  source 1..25: mol_type = other DNA
caaacacacacacacacacacacac

SEQ ID NO: 73 - DNA, 25 bp, synthetic construct
  source 1..25: mol_type = other DNA
cacacacacccacacacacacacac

SEQ ID NO: 74 - DNA, 25 bp, synthetic construct
  source 1..25: mol_type = other DNA
cacacacacacacacccacacacac

SEQ ID NO: 75 - DNA, 25 bp, synthetic construct
  source 1..25: mol_type = other DNA
cacacacacacacacgcacacacac

SEQ ID NO: 76 - DNA, 25 bp, synthetic construct
  source 1..25: mol_type = other DNA
cacacacacacacactcacacacac

SEQ ID NO: 77 - DNA, 25 bp, synthetic construct
  source 1..25: mol_type = other DNA
cacacacacatacacacacacacac

SEQ ID NO: 78 - DNA, 25 bp, synthetic construct
  source 1..25: mol_type = other DNA
cacacacacagacacacacacacac

SEQ ID NO: 79 - DNA, 25 bp, synthetic construct
  source 1..25: mol_type = other DNA
tttacacacacacacacacacacac

SEQ ID NO: 80 - DNA, 25 bp, synthetic construct
  source 1..25: mol_type = other DNA
cacgcacacgcacacacacacacac

SEQ ID NO: 81 - DNA, 25 bp, synthetic construct
  source 1..25: mol_type = other DNA
cacgcacacacgcacacacacgcac

SEQ ID NO: 82 - DNA, 25 bp, synthetic construct
  source 1..25: mol_type = other DNA
cacacgcacacacacacacacacac

SEQ ID NO: 83 - DNA, 20 bp, synthetic construct
  source 1..20: mol_type = other DNA
ccaaatcttataataactac

SEQ ID NO: 84 - RNA, 11 nt, unidentified
  source 1..11: mol_type = transcribed RNA
tgtgtgtgtgt

SEQ ID NO: 85 - RNA, 12 nt, Homo sapiens
  source 1..12: mol_type = transcribed RNA
tgtgtgtgtgtg

SEQ ID NO: 86 - RNA, 13 nt, unidentified
  source 1..13: mol_type = transcribed RNA
tgtgtgtgtgtgt

SEQ ID NO: 87 - RNA, 10 nt, unidentified
  source 1..10: mol_type = transcribed RNA
gtgtgtgtgt

SEQ ID NO: 88 - RNA, 11 nt, unidentified
  source 1..11: mol_type = transcribed RNA
gtgtgtgtgtg

SEQ ID NO: 89 - RNA, 12 nt, unidentified
  source 1..12: mol_type = transcribed RNA
gtgtgtgtgtgt

SEQ ID NO: 90 - RNA, 13 nt, unidentified
  source 1..13: mol_type = transcribed RNA
gtgtgtgtgtgtg

SEQ ID NO: 91 - RNA, 14 nt, unidentified
  source 1..14: mol_type = transcribed RNA
tgtgtgtgtgtgtg

SEQ ID NO: 92 - RNA, 14 nt, unidentified
  source 1..14: mol_type = transcribed RNA
gtgtgtgtgtgtgt

SEQ ID NO: 93 - RNA, 18 nt, unidentified
  source 1..18: mol_type = transcribed RNA
tgtgtgtgtgtgtgtgtg

SEQ ID NO: 94 - RNA, 18 nt, unidentified
  source 1..18: mol_type = transcribed RNA
gtgtgtgtgtgtgtgtgt

SEQ ID NO: 95 - RNA, 10 nt, unidentified
  source 1..10: mol_type = transcribed RNA
tgtgtgtgtg

SEQ ID NO: 96 - DNA, 17 bp, unidentified
  source 1..17: mol_type = unassigned DNA
ggtttgttcagtgtgga

SEQ ID NO: 97 - DNA, 29 bp, unidentified
  source 1..29: mol_type = unassigned DNA
gtgtgtgtgtgtgtgtgtgtgtgtgtgtg

SEQ ID NO: 98 - DNA, 25 bp, unidentified
  source 1..25: mol_type = unassigned DNA
gtgtgtgtgtgtgtgtgtgtgtgtg

SEQ ID NO: 99 - DNA, 20 bp, unidentified
  source 1..20: mol_type = unassigned DNA
gtagttattataagatttgg

SEQ ID NO: 100 - DNA, 21 bp, synthetic construct
  source 1..21: mol_type = other DNA
cagctcatcctcagtcatgtc

SEQ ID NO: 101 - DNA, 21 bp, synthetic construct
  source 1..21: mol_type = other DNA
gatggtgtgactgcaaacttc

SEQ ID NO: 102 - DNA, 23 bp, synthetic construct
  source 1..23: mol_type = other DNA
  misc_feature 1: note = 5HEX linked at the 5' terminus
  misc_feature 23: note = 3IABkFQ linked at the 3' terminus
cagcgccccacaaacacttttct

SEQ ID NO: 103 - DNA, 15 bp, synthetic construct
  source 1..15: mol_type = other DNA
ctgctcagcgtctgc

SEQ ID NO: 104 - DNA, 15 bp, synthetic construct
  source 1..15: mol_type = other DNA
gttgcgaggttccgg

SEQ ID NO: 105 - DNA, 35 bp, synthetic construct
  source 1..35: mol_type = other DNA
  misc_feature 1: note = 5HEX is linked at the 5' terminus
  misc_feature 9^10: note = ZEN between nucleotides 9 and 10
  misc_feature 35: note = 3IABkFQ linked at the 3' terminus
ctaaaacagcaatggcctacaaggaaaaaatgaag

SEQ ID NO: 106 - DNA, 17 bp, synthetic construct
  source 1..17: mol_type = other DNA
cgagccctcggagtttg

SEQ ID NO: 107 - DNA, 20 bp, synthetic construct
  source 1..20: mol_type = other DNA
tccttccaagaaatggtggc

SEQ ID NO: 108 - DNA, 31 bp, synthetic construct
  source 1..31: mol_type = other DNA
  misc_feature 1: note = 56-FAM linked at the 5' terminus
  misc_feature 9^10: note = ZEN between nucleotides 9 and 10
  misc_feature 31: note = 3IABkFQ linked at the 3' terminus
cgacttccagaatatgatgctgctgctgatg

SEQ ID NO: 109 - DNA, 18 bp, synthetic construct
  source 1..18: mol_type = other DNA
tctgagccaaacctgcac

SEQ ID NO: 110 - DNA, 21 bp, synthetic construct
  source 1..21: mol_type = other DNA
atcagctctgtcagcttctcc

SEQ ID NO: 111 - DNA, 21 bp, synthetic construct
  source 1..21: mol_type = other DNA
  misc_feature 1: note = 5HEX linked at the 5' terminus
  misc_feature 9^10: note = ZEN between nucleotides 9 and 10
  misc_feature 21: note = 3IABkFQ linked at the 3' terminus
cgaggctccgtggaacacacg

SEQ ID NO: 112 - DNA, 20 bp, synthetic construct
  source 1..20: mol_type = other DNA
gatcaaaggcgaggagaagg

SEQ ID NO: 113 - DNA, 19 bp, synthetic construct
  source 1..19: mol_type = other DNA
tggcatctgggatcttcac

SEQ ID NO: 114 - DNA, 30 bp, synthetic construct
  source 1..30: mol_type = other DNA
  misc_feature 1: note = 56-FAM linked at 5' terminus
  misc_feature 9^10: note = ZEN between nucleotides 9 and 10
  misc_feature 30: note = 3IABkFQ linked at 3' terminus
acctgtctgcatgagaacctgttccacttc

SEQ ID NO: 115 - DNA, 20 bp, synthetic construct
  source 1..20: mol_type = other DNA
ctgacagtgccaataccacc

SEQ ID NO: 116 - DNA, 19 bp, synthetic construct
  source 1..19: mol_type = other DNA
gctgctccgtggtcttaat

SEQ ID NO: 117 - DNA, 26 bp, synthetic construct
  source 1..26: mol_type = other DNA
  misc_feature 1: note = 5Cy5 linked at 5' terminus
  misc_feature 26: note = 3IAbRQSp linked at 3' terminus
atgaagacgctaaagcccggaagcag

SEQ ID NO: 118 - DNA, 17 bp, synthetic construct
  source 1..17: mol_type = other DNA
  modified_base order(1,2,14,15,16,17): mod_base = OTHER; note = beta-D-oxy-LNA residues
  modified_base 2: mod_base = m5c
  modified_base 16: mod_base = m5c
  modified_base 17: mod_base = m5c
  modified_base 1..17: mod_base = OTHER; note = residues linked by phosphorothioate linkages
tccacactgaacaaacc

SEQ ID NO: 119 - DNA, 29 bp, synthetic construct
  source 1..29: mol_type = other DNA
  modified_base order(1,2,4,6,8,10,12,14,16,18,20,22,24,26,28,29): mod_base = OTHER; note = beta-D-oxy-LNA residues
  modified_base 1: mod_base = m5c
  modified_base 29: mod_base = m5c
  modified_base 1..29: mod_base = OTHER; note = residues linked by phosphorothioate linkages
cacacacacacacacacacacacacacac

SEQ ID NO: 120 - RNA, 25 nt, synthetic construct
  source 1..25: mol_type = other RNA
  misc_feature 1..25: note = 2'-O-methoxyethyl-RNA nucleoside
  modified_base order(1,3,5,7,9,11,13,15,17,19,21,23,25): mod_base = m5c
  modified_base 1..25: mod_base = OTHER; note = residues linked by phosphorothioate linkages
cacacacacacacacacacacacac

SEQ ID NO: 121 - RNA, 29 nt, synthetic construct
  source 1..29: mol_type = other RNA
  misc_feature 1..29: note = 2'-O-methoxyethyl-RNA nucleoside
  modified_base order(1,3,5,7,9,11,13,15,17,19,21,23,25,27,29): mod_base = m5c
  modified_base 1..29: mod_base = OTHER; note = residues linked by phosphorothioate linkages
cacacacacacacacacacacacacacac

SEQ ID NO: 122 - RNA, 25 nt, synthetic construct
  source 1..25: mol_type = other RNA
  misc_feature 1..25: note = 2'-O-methoxyethyl-RNA nucleoside
  modified_base order(1,3,5,7,9,11,13,15,17,19,19,21,23,25): mod_base = m5c
  misc_feature 1: note = CholTEG linked at 5' terminus by a phosphorothioate linkage
  modified_base 1..25: mod_base = OTHER; note = residues linked by phosphorothioate linkages
cacacacacacacacacacacacac

SEQ ID NO: 123 - RNA, 25 nt, synthetic construct
  source 1..25: mol_type = other RNA
  misc_feature 1..25: note = 2'-O-methoxyethyl-RNA nucleoside
  modified_base order(1,3,5,7,9,11,13,15,17,19,21,23,25): mod_base = m5c
  misc_feature 1: note = CholTEG linked at 5' terminus by a phosphate linkage
  modified_base 1..25: mod_base = OTHER; note = residues linked by phosphorothioate linkages
cacacacacacacacacacacacac

SEQ ID NO: 124 - RNA, 29 nt, synthetic construct
  source 1..29: mol_type = other RNA
  modified_base order(1,3,5,7,9,11,13,15,17,19,21,23,25,27,29): mod_base = m5c
  modified_base 1..29: mod_base = OTHER; note = residues linked by phosphorothioate linkages
  misc_feature 1: note = CholTEG linked at 5' terminus by a phosphorothioate linkage
cacacacacacacacacacacacacacac

SEQ ID NO: 125 - RNA, 29 nt, synthetic construct
  source 1..29: mol_type = other RNA
  modified_base 1..29: mod_base = OTHER; note = residues linked by phosphorothioate linkages
  modified_base order(1,3,5,7,9,11,13,15,17,19,21,23,25,27,29): mod_base = m5c
  misc_feature 1: note = CholTEG linked at 5' terminus by a phosphate linker
cacacacacacacacacacacacacacac

SEQ ID NO: 126 - DNA, 20 bp, synthetic construct
  modified_base 1..20: mod_base = OTHER; note = residues linked by phosphorothioate linkages
  source 1..20: mol_type = other DNA
  modified_base order(1,3,4,5,16,17,19,20): mod_base = OTHER; note = beta-D-oxy-LNA residues
  modified_base order(1,17,20): mod_base = m5c
ccaaatcttataataactac